(19) United States
(12) Patent Application Publication
(10) Pub. No.: US 2023/0255556 A1
(43) Pub. Date: Aug. 17, 2023

(54) BIOSENSORS AND FOOD LOGGER SYSTEMS FOR PERSONALIZED HEALTH AND SELF-CARE

(71) Applicant: INFORMED DATA SYSTEMS INC. d/b/a ONE DROP, New York, NY (US)

(72) Inventors: Ydo Wexler, Haifa (IL); Paul Smith, New York, NY (US); Aoife McDonagh, Austin, TX (US); Daniel R. Goldner, Minnetonka, MN (US); Jeffrey Dachis, Brooklyn, NY (US)

(21) Appl. No.: 18/107,933

(22) Filed: Feb. 9, 2023

Related U.S. Application Data
(60) Provisional application No. 63/308,470, filed on Feb. 9, 2022.

(51) Int. Cl. A61B 5/00; G16H 50/30; G16H 15/00; G16H 20/60; A61B 5/0205

(52) U.S. Cl. CPC A61B 5/4866; G16H 50/30; G16H 15/00; G16H 20/60; A61B 5/0205; A61B 5/7267; A61B 5/7275; A61B 5/7282; A61B 5/486; A61B 5/14532

(57) ABSTRACT

Systems and methods for food logging are disclosed herein. In some embodiments, a food logging system converts text inputs that describe an instance of dietary intake in the form of unstructured text to a detailed description of the nutritional content of the instance in the form of structured data. The food logging system can consist of five structural components, each of which performs a specific task. Each food logging system component is associated with a defined resource that provides information and directives on how the associated component is to perform its designated task. Together, these components and associated resources constitute an apparatus that enables users to carry out detailed and accurate food logging, simply by providing textual descriptions of their dietary intake.

### CROSS-REFERENCE TO RELATED APPLICATION

The present application claims the benefit of U.S. Provisional Patent Application No. 63/308,470, filed Feb. 9, 2022, entitled FOOD LOGGER SYSTEMS FOR PERSONALIZED HEALTH AND SELF-CARE, AND ASSOCIATED METHODS, which is incorporated by reference herein in its entirety.

### TECHNICAL FIELD

This disclosure relates generally to biosensor systems, food loggers, and personalized healthcare and, in particular, to systems and methods for food logging.

### BACKGROUND

Several factors often limit users' adherence to food logging because current logging tools can be cumbersome, tedious, and slow. Food logging users regularly face challenges, such as every food item must be searched individually in a nutrition database. In many cases, a user needs to look through many possible results and their corresponding nutrition profiles to decide which result is most suitable. There can be uncertainty in selecting the best match from the results provided by a nutrition database. Such difficulties arise for several reasons, including if the food is home-cooked, or if it is an obscure food. A user must translate portion sizes provided by a nutrition database to portions representing what the user ate. Many food databases store food entries with inconsistent unit types. There is generally no standardization in the use of either metric or imperial units. Therefore, the unit a user measures their food may not always be available. Additionally, these calculations invite the possibility of human error both in performing the mental arithmetic and transcribing of results.

### BRIEF DESCRIPTION OF THE DRAWINGS

FIG. 1 is a schematic diagram illustrating an exemplary computing environment in which a healthcare guidance system operates, in accordance with embodiments of the present technology.

FIG. 2 is a flowchart illustrating a process of a food logging system, in accordance with embodiments of the present technology.

FIG. 3 is a flowchart illustrating a process for converting text into pairs of quantity and food terms, in accordance with embodiments of the present technology.

FIG. 4 is a flowchart illustrating a process for acquiring optimal nutrition information for each input, in accordance with embodiments of the present technology.

FIG. 5 is a flowchart illustrating a process for culling and ranking nutrition information mappings, in accordance with embodiments of the present technology.

FIG. 6 is a diagram illustrating selecting an appropriate serving description from those available in the result chosen from the nutrition database, in accordance with embodiments of the present technology.

FIG. 7 is a flowchart illustrating a process for selecting a serving description, in accordance with embodiments of the present technology.

FIG. 8 diagram illustrating storing information related to a food logging instance, in accordance with embodiments of the present technology.

FIG. 9 is a flowchart illustrating a process for calculating a nutrient value based on the information extracted by a characteristic food language processor, in accordance with embodiments of the present technology.

FIG. 10 is a flowchart illustrating a process for calculating liquid volume for a food item based on the information extracted by the characteristic food language processor, in accordance with embodiments of the present technology.

FIG. 11 is a graph illustrating food logging accuracy for annotated meals, in accordance with embodiments of the present technology.

FIG. 12 illustrates an example of a food logging session, in accordance with embodiments of the present technology.

FIG. 13 is a schematic block diagram of a computing system or device configured in accordance with embodiments of the present technology.

FIGS. 14 and 15 are schematic diagrams illustrating exemplary computing environments in which a healthcare guidance system operates, in accordance with embodiments of the present technology.

FIG. 16 is a flowchart illustrating a process for providing personalized health information, in accordance with embodiments of the present technology.

FIG. 17 is a flowchart illustrating a process for identifying condition-specific adverse events for dietary health, in accordance with embodiments of the present technology.

FIG. 18 illustrates an example of a user interface displaying health information, in accordance with embodiments of the present technology.

### DETAILED DESCRIPTION

The present technology generally relates to systems and methods for converting text inputs, that describe an instance of dietary intake in the form of unstructured text, to a detailed description of the nutritional content of the instance in the form of structured data. The food logging system can incorporate a natural language processing (NLP) system tailored for the food logging. The food logging system can extract information relevant to estimating nutrient values from unconstrained user text, namely, food items and their associated portions. The extracted information is used to make database queries and automate the steps that a user would perform when logging food. The system automates all portion conversions and calculations required to reach a final estimate of the nutrient content of a user's meal.

The food logging system develops a solution for extracting nutrition information from unconstrained text descriptions. The food logging system employs NLP methods to extract food and quantity words from user inputted text. The extracted food words are used to query a nutrition database (e.g., FatSecret or USDA) for nutrition information. The extracted portion words are used to scale the nutrient values provided by the database to match the user's specified portion. The nutrition information estimated by the food logging system is shown to the user in a simple text-based format. Further iterations of this system can store logged nutrition information to the user's account for subsequent processing and insight generation.

The food logging system can increase user participation in food logging. Food logging can generate a method of self-care for individuals living with chronic conditions. It is often prescribed by dietitians to people newly diagnosed with diabetes. User participation can be increased by tackling the barriers to food logging that currently exist. The downstream effects of increasing user participation in food logging include receiving larger quantities of food logging data from users and the generation of valuable insights from these data.

The food logging system can provide benefits to users. The practice of consistently logging meals can build an awareness of food consumption habits, making it an important component of diet management, especially for individuals with chronic conditions. Awareness of one's food consumption habits is of such importance that when someone is first diagnosed with diabetes of any type, they will typically be assigned the task of food logging by a dietician. By making food logging easier, a user is more likely to continue the practice and reap the commensurate benefits.

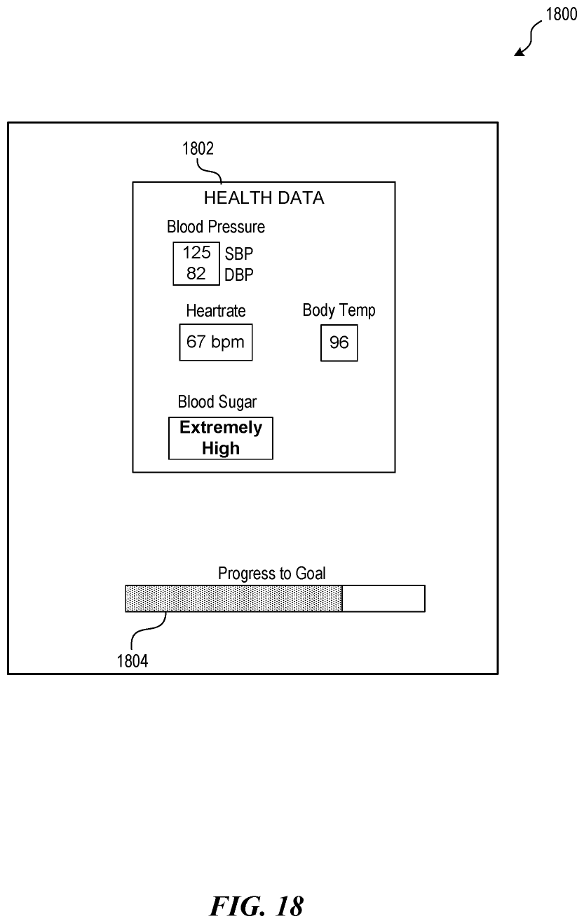

Beyond simply increasing user participation in food logging, the food logging system can provide more comprehensive nutrition information than that obtained through manual logging. For example, if the portion sizes of foods are not considered accurately, if condiments that dress a meal are forgotten, or if beverages are omitted, much of the accuracy (thus utility) of food logging is lost. By virtue of its thoroughness, the food logging system avoids such errors. Another benefit of food logging for users is that it can help identify how food affects chronic conditions such as diabetes, hypertension and heart disease. While over time, individuals may become aware of the effects of foods on their state of wellbeing, they may not be aware of all foods which can exacerbate their symptoms. As such, the food logging system paired with symptom logging could be used to identify foods previously unknown to affect one's chronic condition. Participating in food logging via a user device (e.g., smartphone, laptop, tablet, smartwatch, etc.) will deliver users from much of the burden of remembering what they're eating over time. Such assistance can be extremely beneficial to users who are asked by physicians to fill out food frequency questionnaires as part of treatment for a chronic condition. The food logging system can provide progress information (e.g., progress toward one or more goals, percentage completion progress, etc.), reminders, notifications, alerts, and other information via an interface (e.g., GUI), audible feedback, tactile notifications, or the like. For example, a user interface 1800 in FIG. 18 illustrates a progress indicator 1804. The food logging system can interact with one or more user devices to manage personalized care. The food logging system can authorize, authenticate, pair with, and/or control acquisition and/or communication of dietary related data (e.g., analyte levels associated with dietary action).

Implementing the food logging system will offer numerous benefits to healthcare databases, as the food logging system will generate a large volume of currently scarce data points. Food logging data generated by the food logging system can offer insight to users, as there is a large need for such insights. Understanding how food affects chronic conditions is of high importance to individuals affected by them. For example, nutrient specific insights might be important to someone living with diabetes trying to control their blood sugar. Food logging data can also be a rich source of insights from a behavioral science perspective. For example, useful insights can be generated on an individual's snacking habits and the relationship between these habits and variables such as snacking frequency, associated locations, and times of day can help provide insights about phenomena such as eating to cope with stress. Armed with such insights, the food logging system can offer effective user interaction to promote healthier eating. The food logging system can track the user's location (e.g., via positioning information from a mobile device, wearable device, user input), stress levels (e.g., based on heart rate or other stress indicators), vitals (e.g., condition-related vitals), analyte levels (e.g., condition-related vitals), historical eating patterns, blood levels, weight, and/or additional information to predict whether a user may be prone to dietary events, such as eating or drinking. The food logging system can then provide output to the user based on, for example, user-goals, medical restrictions, healthcare input, etc. The output can be based on predicted user-specific behavioral responses, thereby achieving targeted dietary outcomes correlated to healthier dietary action. The output can include predictions (e.g., predicted condition-specific adverse events, alteration of metabolic state, weight gain/loss, hypoglycemia, hyperglycemia, pre-diabetes, hypertension, hyperlipidemia, ketoacidosis, etc.) warnings, alerts, or the like. The predicted condition-specific adverse events can be, for example, hypoglycemia events or hyperglycemia events for a diabetic user.

Embodiments of the present disclosure will be described more fully hereinafter with reference to the accompanying drawings in which numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology.

Systems for Food Logging and Healthcare Guidance

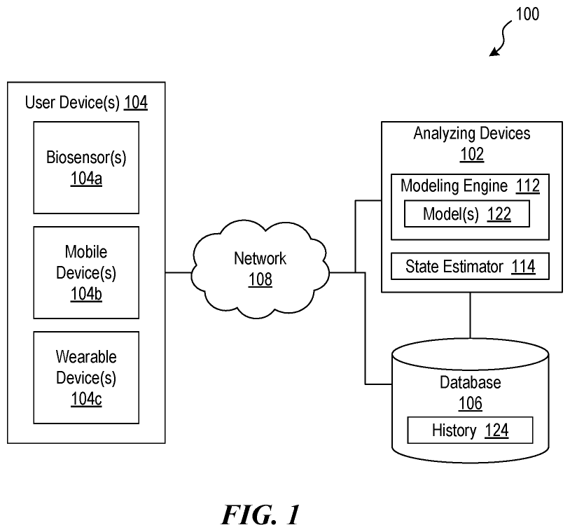

FIG. 1 is a schematic diagram of an exemplary computing environment in which a biomonitoring and healthcare guidance system 100 (“system 100”) operates, in accordance with embodiments of the present technology. As shown in FIG. 1, the system 100 (e.g., food logging system) can include one or more analyzing devices, one or more user devices 104, and/or at least one database or storage component 106 (“database 106”) operably coupled to each other, such as via a network 108. The system 100 is also operably coupled to at least one database or storage component 106 (“database 106”). The system 100 can include processors, memory, and/or other software and/or hardware components configured to implement the various methods described herein. For example, the system 100 can be configured to log food information, monitor a patient/user health state, and provide predictive self-care guidance, as described in greater detail below.

For example, the user devices 104 can obtain biometric data, such as temperature, heartrate, blood pressure, blood glucose level or the like, and/or spatial data, such as location, acceleration, velocity, orientation, a change thereof over time, or the like. The user devices 104 can also obtain contextual data, such as user calendar (including, e.g., event name or category, location, date, time, participant, etc.), that may be used to categorize other obtained data. For example, the contextual data may be used to categorize the data into user activities or user health states.

The health state can be any status, condition, parameter, etc. that is associated with or otherwise related to the patient's health. In some embodiments, the system 100 can be used to identify, manage, monitor, and/or provide recommendations relating to diabetes, hypoglycemia, hyperglycemia, pre-diabetes, hypertension, hyperlipidemia, ketoacidosis, liver failure, congestive heart failure, coronary artery disease, myocardial infarction, ischemic stroke, hypoxia, kidney function, chronic kidney disease, intoxication, dehydration, hyponatremia, shock, sepsis, trauma, water retention, bleeding, endocrine disorders, asthma, lung conditions, muscle breakdown, malnutrition, body function (e.g., lung functions, heart functions, etc.), physical performance (e.g., athletic performance), anaerobic activity, weight loss/gain, nutrition, wellness, sleep disorder, mental health, focus, effects of medication, medication levels, health indicators, and/or user compliance. In some embodiments, the system 100 receives input data and performs monitoring, processing, analysis, forecasting, interpretation, etc. of the input data in order to generate instructions, notifications, recommendations, support, and/or other information to the patient that may be useful for self-care of diseases or conditions, such as chronic conditions (e.g., diabetes (type 1 and type 2), pre-diabetes, hypertension, hyperlipidemia, etc.).

The input data for the system 100 can include food information, dietary information, health-related information, contextual information, and/or any other information relevant to the patient's health state. For example, health-related information can include levels or concentrations of a biomarker, such as glucose, electrolytes, neurotransmitters, amino acids, hormones, alcohols, gases (e.g., oxygen, carbon dioxide, etc.), creatinine, blood urea nitrogen (BUN), lactic acid, drugs, pH, cell count, and/or other biomarkers. Health-related information can also include physiological and/or behavioral parameters, such as vitals (e.g., heart rate, body temperature (such as skin temperature), blood pressure (such as systolic and/or diastolic blood pressure), respiratory rate), cardiovascular data (e.g., pacemaker data, arrhythmia data), body function data, meal or nutrition data (e.g., number of meals; timing of meals; number of calories; amount of carbohydrates, fats, sugars, etc.), physical activity or exercise data (e.g., time and/or duration of activity; activity type such as walking, running, swimming; strenuousness of the activity such as low, moderate, high; etc.), sleep data (e.g., number of hours of sleep, average hours of sleep, variability of hours of sleep, sleep-wake cycle data, data related to sleep apnea events, sleep fragmentation (such as fraction of nighttime hours awake between sleep episodes, etc.), stress level data (e.g., cortisol and/or other chemical indicators of stress levels, perspiration), al c data, etc. Health-related information can also include medical history data (e.g., weight, age, sleeping patterns, medical conditions, cholesterol levels, disease type, family history, patient health history, diagnoses, tobacco usage, alcohol usage, etc.), diagnostic data (e.g., molecular diagnostics, imaging), medication data (e.g., timing and/or dosages of medications such as insulin), personal data (e.g., name, gender, demographics, social network information, etc.), and/or any other data, and/or any combination thereof. Contextual information can include user location (e.g., GPS coordinates, elevation data), environmental conditions (e.g., air pressure, humidity, temperature, air quality, etc.), and/or combinations thereof.

In some embodiments, a guidance system or analyzing devices 102 (“analyzing device 102”) receive the input data from one or more user devices 104. The user devices 104 can be any device associated with a patient or other user, and can be used to obtain patient data, condition of patient, historical food-related outcomes/effects, food information, food/drink quantity information (e.g., images of food, mass/weight information from digital scales, etc.), healthcare information, contextual information, and/or any other relevant information relating to the patient and/or any other users or patients (e.g., appropriately anonymized patient data). In the illustrated embodiment, for example, the user devices 104 can include at least one biosensor 104a (e.g., blood glucose sensors, pressure sensors, heart rate sensors, sleep trackers, temperature sensors, motion sensors, or other biomonitoring devices), at least one mobile device 104b (e.g., a smartphone or tablet computer), and, optionally, at least one wearable device 104c (e.g., a smartwatch, fitness tracker). In other embodiments, however, one or more of the devices 104a-c can be omitted and/or other types of user devices can be included, such as computing devices (e.g., personal computers, laptop computers, etc.). Additionally, although FIG. 1 illustrates the biosensor(s) 104a as being separate from the other user devices 104, in other embodiments the biosensor(s) 104a can be incorporated into another user device 104.

The biosensor 104a can include various types of sensors, such as chemical sensors, electrochemical sensors, optical sensors (e.g., optical enzymatic sensors, opto-chemical sensors, fluorescence-based sensors, etc.), spectrophotometric sensors, spectroscopic sensors, polarimetric sensors, calorimetric sensors, iontophoretic sensors, radiometric sensors, and the like, and combinations thereof. In some embodiments, the biosensor 104a is or includes a blood glucose sensor. The blood glucose sensor can be any device capable of obtaining blood glucose data from the patient, such as implanted sensors, non-implanted sensors, invasive sensors, minimally invasive sensors, non-invasive sensors, wearable sensors, etc. The blood glucose sensor can be configured to obtain samples from the patient (e.g., blood samples) and determine glucose levels in the sample. Any suitable technique for obtaining patient samples and/or determining glucose levels in the samples can be used. In some embodiments, for example, the blood glucose sensor can be configured to detect substances (e.g., a substance indicative of glucose levels), measure a concentration of glucose, and/or measure another substance indicative of the concentration of glucose. The blood glucose sensor can be configured to analyze, for example, body fluids (e.g., blood, interstitial fluid, sweat, etc.), tissue (e.g., optical characteristics of body structures, anatomical features, skin, or body fluids), and/or vitals (e.g., heat rate, blood pressure, etc.) to periodically or continuously obtain blood glucose data. Optionally, the blood glucose sensor can include other capabilities, such as processing, transmitting, receiving, and/or other computing capabilities. In some embodiments, the blood glucose sensor can include at least one continuous glucose monitoring (CGM) device or sensor that measures the patient's blood glucose level at predetermined time intervals. For example, the CGM device can obtain at least one blood glucose measurement every minute, 2 minutes, 5 minutes, 10 minutes, 15 minutes, 20 minutes, 30 minutes, 60 minutes, 2 hours, etc. In some embodiments, the time interval is within a range from 5 minutes to 10 minutes.

In some embodiments, some or all of the user devices 104 may be configured to continuously obtain any of the above data (e.g., health-related information and/or contextual information) from the patient over a particular time period (e.g., hours, days, weeks, months, years). For example, data can be obtained at a predetermined time interval (e.g., e.g., in the order of seconds, minutes, or hours), at random time intervals, or combinations thereof. The time interval for data collection can be set by the patient, by another user (e.g., a physician), by the analyzing devices 102, or by the user device 104 itself (e.g., as part of an automated data collection program). The user device 104 can obtain the data automatically or semi-automatically (e.g., by automatically prompting the patient to provide such data at a particular time), or from manual input by the patient (e.g., without prompts from the user device 104). The continuous data may be obtained by the system (e.g., collected at the analyzing devices 102) at predetermined time intervals (e.g., once every minute, 2 minutes, 5 minutes, 10 minutes, 15 minutes, 20 minutes, 30 minutes, 60 minutes, 2 hours, etc.), continuously, in real-time, upon receiving a query, manually, automatically (e.g., upon detection of new data), semi-automatically, etc. The time interval at which the user device 104 obtains data may or may not be the same as the time interval at which the user device 104 transmits the data to the analyzing devices 102.

The food logging system 100 can control operation of the user device(s) 104 based on, at least in part, the user input (e.g., user inputted confidences, accuracy settings, etc.), user condition, risk of adverse event, healthcare provider, etc. For example, the food logging system 100 can increase the frequency of sampling performed by the user device 104 if the user is prone to experience food-related adverse events. In some embodiments, the food logging system 100 can set a high data acquisition rate (e.g., analytes sampling rate) of the user device 104 of a user with type one diabetes and a lower sampling rate for a user with type 2 diabetes. The acquisition rate can also be selected based on food related events. For example, if the user typically consumes (e.g., eats or drinks) food at certain times, the food logging system 100 can increase sampling rates immediately prior, during, and/or after scheduled food related events.

The food logging system 100 can determine correlations between the user's activity/location and dietary actions. In some embodiments, the food logging system 100 evaluates location data of the user. The location data can be correlated to potential food events. For example, in response to the user checking into or being located at a restaurant, the food logging system 100 can perform one or more pre-meal routines to provide dietary suggestions, track the dietary effects, provide current/predicted goal progress, etc. In some embodiments, one or more notifications or alerts can be sent to the user to assist with ordering food. The notifications or alerts can include, for example, biometric data, historical biometric data, predicted food-related outcomes (e.g., hypoglycemic or hyperglycemic events, maintenance of metabolic state such as ketosis, etc.). In response to the user checking into or being located at a grocery store, the food logging system 100 can send one or more shopping recommendations for healthy eating. The current biometric data can be evaluated to generate the shopping recommendations based on the user's specific condition.

The user devices 104 can obtain any of the above data and can provide output in various ways, such as using one or more of the following components: a microphone (either a separate microphone or a microphone imbedded in the device), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, a sensor (e.g., a sensor included in or operably coupled to the user device 104), and/or any other device. The data obtained by the user devices 104 can include metadata, structured content data, unstructured content data, embedded data, nested data, hard disk data, memory card data, cellular telephone memory data, smartphone memory data, main memory images and/or data, forensic containers, zip files, files, memory images, and/or any other data/information. The data can be in various formats, such as text, numerical, alpha-numerical, hierarchically arranged data, table data, email messages, text files, video, audio, graphics, etc. Optionally, any of the above data can be filtered, smoothed, augmented, annotated, or otherwise processed (e.g., by the user devices 104 and/or the analyzing devices 102) before being used.

In some embodiments, any of the above data can be queried by one or more of the user devices 104 from one or more databases (e.g., the database 106, a third-party database, etc.). The user device 104 can generate a query and transmit the query to the analyzing devices 102, which can determine which database may contain requisite information and then connect with that database to execute a query and retrieve appropriate information. In other embodiments, the user device 104 can receive the data directly from the third-party database and transmit the received data to the analyzing devices 102, or can instruct the third-party database to transmit the data to the analyzing devices 102. In some embodiments, the analyzing devices 102 can include various application programming interfaces (APIs) and/or communication interfaces that can allow interfacing between user devices 104, databases, and/or any other components.

Optionally, the analyzing devices 102 can also obtain any of the above data from various third-party sources, e.g., with or without a query initiated by a user device 104. In some embodiments, the analyzing devices 102 can be communicatively coupled to various public and/or private databases that can store various information, such as census information, food information, nutrient information, health statistics (e.g., appropriately anonymized), demographic information, population information, and/or any other information. Additionally, the analyzing devices 102 can also execute a query or other command to obtain data from the user devices 104 and/or access data stored in the database 106. The data can include data related to the particular patient and/or a plurality of patients or other users (e.g., health-related information, contextual information, etc.) as described herein.

The database 106 can be used to store various types of data obtained and/or used by the analyzing devices 102. For example, any of the above data can be stored as user history 124 in the database 106. The database 106 can also be used to store data generated by the system 100, such as previous predictions or forecasts produced by the system 100. In some embodiments, the database 106 includes data for multiple users, such as a plurality of patients (e.g., at least 50, 100, 200, 500, 1000, 2000, 3000, 4000, 5000, or 10,000 different patients). The data can be appropriately anonymized to ensure compliance with various privacy standards. The database 106 can store information in various formats, such as table format, column-row format, key-value format, etc. (e.g., each key can be indicative of various attributes associated with the user and each corresponding value can be indicative of the attribute's value (e.g., measurement, time, etc.)). In some embodiments, the database 106 can store a plurality of tables that can be accessed through queries generated by the analyzing devices 102 and/or the user devices 104. The tables can store different types of information (e.g., one table can store blood glucose measurement data, another table can store user health data, etc.), where one table can be updated as a result of an update to another table.

In some implementations, the system 100 can collect and analyze periodically (e.g., every second, every minute, hourly, daily, weekly, monthly, etc.) users' health data (e.g., dietary data). In some embodiments, the system can determine one or more collection rates based on food logging, user activity, user location, time of day, collected biometric data, and/or other data disclosed herein. The system 100 can identify (e.g., forecast) a health event (e.g., high or low blood pressure, high or low blood glucose levels, risk of cardiovascular disease, etc.) of the user based on the health data. The system 100 can select (or generate) a self-care mode with an action or sequence of actions (e.g., exercise, diet, sleep, reduce stress, etc.) for the user to perform to mitigate the risk of the health event or avoid the health event. The user can receive a notification (e.g., SMS message, email, alert on a user interface, etc.) which includes the forecasted health event, the actions to perform to mitigate or avoid the health event, and the self-care mode. For example, the system 100 can determine that the blood pressure of the user is too high and forecast that the user can experience a heart attack or a stroke if no user action is taken to lower the blood pressure. The system 100 can select a self-care mode which contains an action or sequence of actions directed towards lowering the blood pressure of the user. The user can receive a notification alerting them of their high blood pressure, the forecasted cardiovascular risk, and actions for the user to perform to avoid the adverse cardiovascular complications. The food logging system can correlate dietary actions with the notification. In some embodiments, the notifications can include dietary actions to manage high blood pressure, forecasted cardiovascular risk, and/or actions for the user to perform to avoid the adverse events or complications (e.g., cardiovascular-related complications). In some cases, the notification can include a recommendation to seek medical assistance based on the health data.

In some embodiments, one or more users can access the system 100 via the user devices 104, e.g., to send data to the analyzing devices 102 (e.g., food information, health-related information, contextual information) and/or receive data from the system 100 (e.g., predictions, notifications, recommendations, instructions, support, etc.). The users can be individual users (e.g., patients, healthcare professionals, etc.), computing devices, software applications, objects, functions, and/or any other types of users and/or any combination thereof. For example, upon obtaining any of the input data discussed above, the user device 104 can generate an instruction and/or command to the analyzing devices 102, e.g., to process the obtained data, store the data in the database 106, extract additional data from one or more databases, and/or perform analysis of the data. The instruction/command can be in a form of a query, a function call, and/or any other type of instruction/command. In some implementations, the instructions/commands can be provided using a microphone (either a separate microphone or a microphone imbedded in the user device 104), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, and/or using any other device. The user device 104 can also instruct the analyzing devices 102 to perform an analysis of data stored in the database 106 and/or inputted via the user device 104.

As discussed further below, the analyzing devices 102 can analyze the obtained input data, including historical data, current real-time data, continuously supplied data, and/or any other data (e.g., using a statistical analysis, machine learning analysis, etc.), and generate output data. The output data can include predictions of a patient's health state, interpretations, recommendations, notifications, instructions, support, and/or other information related to the obtained input data. The analyzing devices 102 can perform such analyses at any suitable frequency and/or any suitable number of times (e.g., once, multiple times, on a continuous basis, etc.). For example, when updated input data is supplied to the analyzing devices 102 (e.g., from the user devices 104), the analyzing devices 102 can reassess and update its previous output data, if appropriate. In performing its analysis, the analyzing devices 102 can also generate additional queries to obtain further information (e.g., from the user devices 104, the database 106, or third-party sources). In some embodiments, the user device 104 can automatically supply the analyzing devices 102 with such information. Receipt of updated/additional information can automatically trigger the analyzing devices 102 to execute a process for reanalyzing, reassessing, or otherwise updating previous output data.

In some embodiments, the analyzing devices 102 is configured to analyze the input data and generate the output data using one or more machine learning models 122. The machine learning models 122 can include supervised learning models, unsupervised learning models, semi-supervised learning models, and/or reinforcement learning models generated by one or more modeling engines 112. Examples of machine learning models suitable for use with the present technology include, but are not limited to: regression algorithms (e.g., ordinary least squares regression, linear regression, logistic regression, stepwise regression, multivariate adaptive regression splines, locally estimated scatterplot smoothing), instance-based algorithms (e.g., k-nearest neighbor, learning vector quantization, self-organizing map, locally weighted learning, support vector machines), regularization algorithms (e.g., ridge regression, least absolute shrinkage and selection operator, elastic net, least-angle regression), decision tree algorithms (e.g., classification and regression trees, Iterative Dichotomiser 3 (ID3), C4.5, C5.0, chi-squared automatic interaction detection, decision stump, M5, conditional decision trees), Bayesian algorithms (e.g., naïve Bayes, Gaussian naïve Bayes, multinomial naïve Bayes, averaged one-dependence estimators, Bayesian belief networks, Bayesian networks), clustering algorithms (e.g., k-means, k-medians, expectation maximization, hierarchical clustering), association rule learning algorithms (e.g., apriori algorithm, ECLAT algorithm), artificial neural networks and deep learning networks (e.g., perceptron, multilayer perceptrons, convolutional networks, residual networks, attention-based networks, transformers, probabilistic times series networks, wavelet networks, Generative Pre-trained Transformers (GPT), adversarial networks, generative adversarial networks, Hopfield networks, radial basis function networks, recurrent neural networks, long short-term memory networks, stacked auto-encoders, deep Boltzmann machines, deep belief networks), network optimization algorithms (e.g., back-propagation, stochastic gradient descent, feed-forward optimization), dimensionality reduction algorithms (e.g., principal component analysis, principal component regression, kernel principal component analysis, partial least squares regression, Sammon mapping, multidimensional scaling, projection pursuit, discriminant analysis, independent component analysis, non-negative matrix factorization, truncated singular value decomposition, latent Dirichlet allocation), time series forecasting algorithms (e.g., exponential smoothing, autoregressive models, autoregressive with exogenous input (ARX) models, autoregressive moving average (ARMA) models, autoregressive moving average with exogenous inputs (ARMAX) models, autoregressive integrated moving average (ARIMA) models, autoregressive conditional heteroskedasticity (ARCH) models, Holt-Winters algorithm), and ensemble algorithms (e.g., boosting, bootstrapped aggregation, AdaBoost, blending, stacking, gradient boosting machines, gradient boosted trees, random forest, bagging, voting).

Although FIG. 1 illustrates a single set of user devices 104, it will be appreciated that the analyzing devices 102 can be operably and communicably coupled to multiple sets of user devices, each set being associated with a particular patient or user. Accordingly, the system 100 can be configured to receive and analyze data from a large number of patients (e.g., at least 50, 100, 200, 500, 1000, 2000, 3000, 4000, 5000, or any number of different patients) over an extended time period (e.g., weeks, months, years). The data from these patients can be used to train and/or refine one or more machine learning models implemented by the analyzing devices 102, as described below.

The analyzing devices 102 and user devices 104 can be operably and communicatively coupled to each other via the network 108. The network 108 can be or include one or more communications networks, and can include at least one of the following: a wired network, a wireless network, a metropolitan area network (“MAN”), a local area network (“LAN”), a wide area network (“WAN”), a virtual local area network (“VLAN”), an internet, an extranet, an intranet, and/or any other type of network and/or any combination thereof. Additionally, although FIG. 1 illustrates the analyzing devices 102 as being directly connected to the database 106 without the network 108, in other embodiments the analyzing devices 102 can be indirectly connected to the database 106 via the network 108. Moreover, in other embodiments one or more of the user devices 104 can be configured to communicate directly with the analyzing devices 102 and/or database 106, rather than communicating with these components via the network 108.

The various components 102-108 illustrated in FIG. 1 can include any suitable combination of hardware and/or software. In some embodiment, components 102-108 can be disposed on one or more computing devices, such as, server(s), database(s), personal computer(s), laptop(s), cellular telephone(s), smartphone(s), tablet computer(s), and/or any other computing devices and/or any combination thereof. In some embodiments, the components 102-108 can be disposed on a single computing device and/or can be part of a single communications network. Alternatively, the components can be located on distinct and separate computing devices. For example, although FIG. 1 illustrates the analyzing devices 102 as being a single component, in other embodiments the analyzing devices 102 can be implemented across a plurality of different hardware components at different locations.

In some embodiments, the analyzing devices 102 may include a state estimator 114 configured to estimate a user context or a user health state. The state estimator 114 can use the above-described input data to determine a current or ongoing activity or state of the user. Similarly, the state estimator 114 can analyze the above-described input data to predict a future activity or health state of the user. The state estimator 114 can use corresponding machine learning models 122 to generate the estimated user states. For example, the state estimator 114 can use the models 122 to predict based on the current state and the user history 124 that blood glucose levels will reach a threshold level at a time when the user will likely be incapacitated (e.g., sleeping or intoxicated). In some embodiments, the state estimator 114 can generate activity predictions for a predetermined future duration based on the obtained data. The state estimator 114 can start from a current health state (e.g., blood glucose level) and extrapolate or derive future health states according to the activity predictions. The system 100 can use the future health states to determine and recommend a set of user actions that may be implemented between now and a future time to avoid the thresholding health states and/or to conform to a targeted behavior (e.g., a repeated set of user actions).

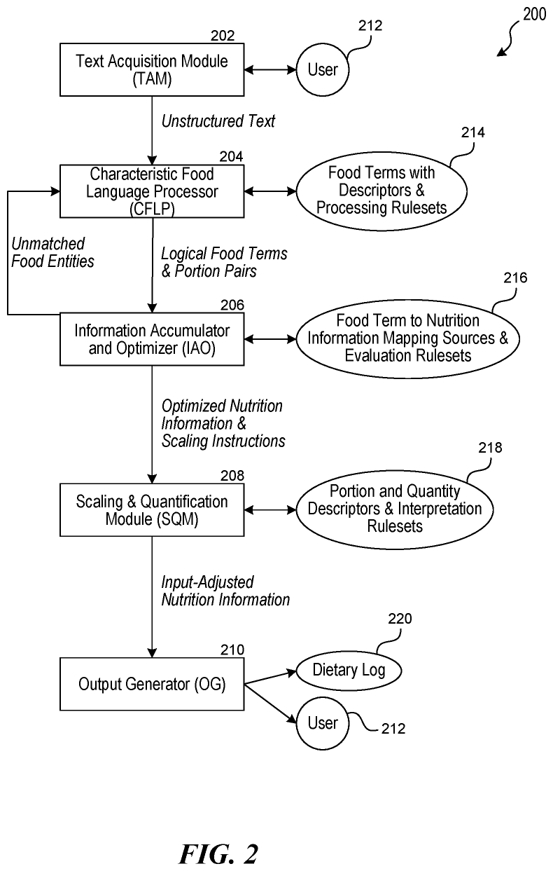

FIG. 2 is a block diagram illustrating a process 200 of a food logger system, in accordance with embodiments of the present technology. The food logging system consists of five structural components, each of which performs a specific task. Each food logging system component is associated with a defined resource that provides information and directives on how the associated component is to perform its designated task. Together, these components and associated resources constitute an effective apparatus that enables users to carry out detailed and accurate food logging simply by providing textual descriptions of their dietary intake.

At step 202, the food logger system receives textual input from a user 212 describing food and/or beverages consumed. Consumed can refer to food and/or beverages that the user has consumed or may consume. The Text Acquisition Module (TAM) obtains unstructured text describing food and beverages consumed through its interaction with a user 212. The input can pertain to a single sitting or meal, or several thereof. In the current implementation, TAM functionality can receive as input user typed text or speech which is then translated to text. As configured, TAM elicits both a term indicating a meal (e.g., “lunch”) and one or more words corresponding to a food item. Once both have been provided by the user 212, the unstructured text is sent as output (e.g., via a JSON string) to the Characteristic Food Language Processor (CFLP).

At step 204, the food logger system converts the unstructured text input to logical pairings of text phrases describing a single food/beverage item (termed “food entity” herein) and the text describing the serving, portion, or amount associated with this entity, such as food terms with descriptors and processing rulesets 214. The CFLP component of the food logging system takes as input unstructured text containing a meal description including specific foods and beverages consumed as well as amounts of each and generates as output data objects representing individual food entities and their respective quantifiers. These data objects contain a substring from the original input identified as corresponding to a single food entity recognizable by the mapping resources of the Information Accumulator and Optimizer (IAO) component as well as all terms identified as quantifiers for the given food entity. For example:

Input (Unstructured Text):
“For lunch, I ate two big bowls of brown rice”

Output (Paired Food Entity and Quantifier Strings):
Food entity string: “brown rice”
Quantifier string “two big bowls”

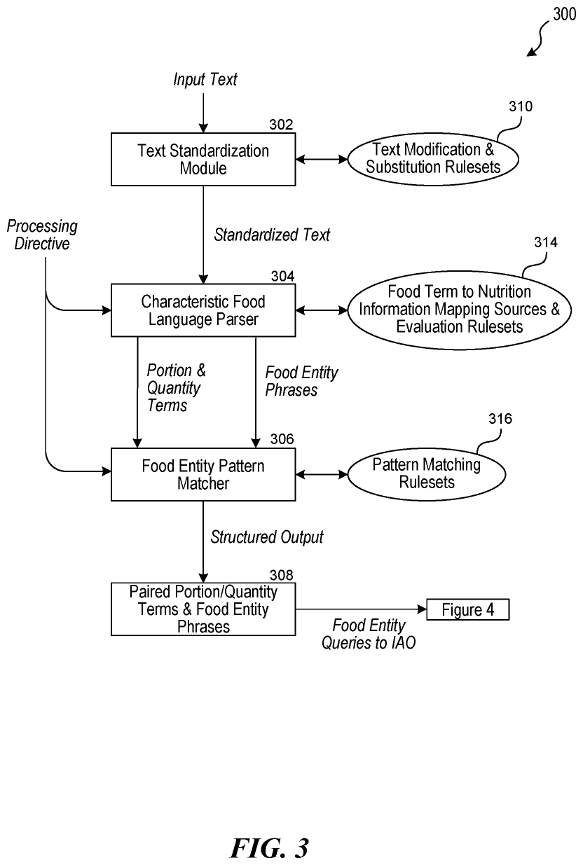

In order to generate a list of such data objects from unstructured text input containing an arbitrary number of food entities and quantifiers, the CFLP employs three sub-components as shown in FIG. 3 and described as follows: 1) a text standardization module that transforms unstructured text input by a fixed set of rules, 2) a parsing module that extracts and categorizes all words in the standardized text input as either explicitly a food entity, a food-related word, a quantifier, an important conjunction, or a stray/uninformative word, and 3) a pattern matching module that identifies and labels the syntactic organization of food entities and conjunctions for a given demarcated component of a meal description. Each of these sub-components relies on an external set of word lists, processing directives, or rulesets.

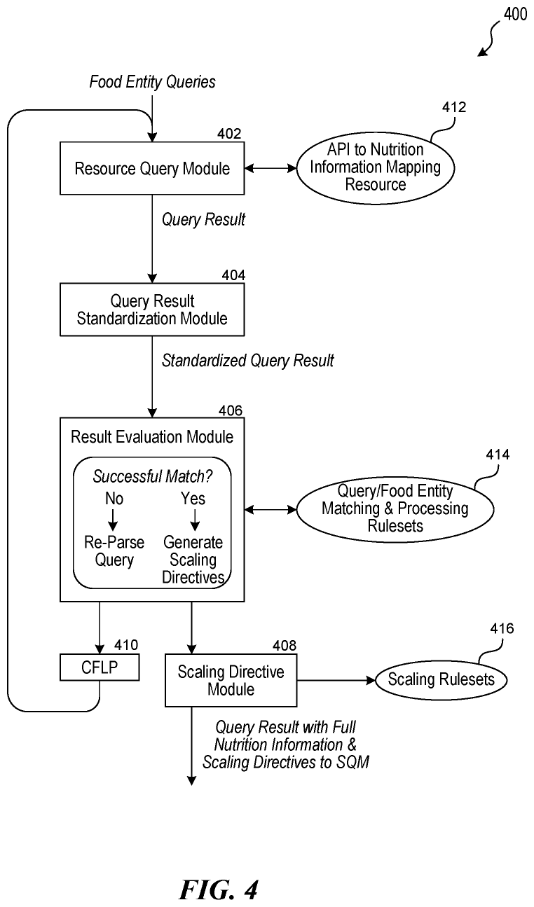

At step 206, the food logger system acquires nutrition information for these food entities. The food logger system can acquire the nutrition information from food term to nutrition information mapping sources and evaluation rulesets 216. The IAO component of the food logging system takes as input a single quantifier/food entity pair produced by the CFLP and generates as output a data object containing both this pair and one or more nutrition information mappings for the food entity selected as the most optimal from all made available from external resources. To accomplish this task, the IAO incorporates four distinct sub-components as shown in FIG. 4 and described as follows: 1) a resource query module (step 402) that acquires nutrition information mappings that describe the amount of macronutrients in of a given portion of a given food entity; 2) a standardization module (step 404) that converts these mappings to a standard format; 3) a result evaluation module (step 406) that both selects the most optimal nutrition information mapping from all those made available and decides if the input query needs re-parsed by the CLFP; and 4) a scaling directive module (step 408) that, if necessary, uses the pattern determined by the CLFP to adjust portion scales and amounts. In addition, the result evaluation module can also examine portion and serving information acquired from external sources to both, discard nutrition information mappings representing outliers, and ensure that each and every query is paired with at least one nutrition information mapping defined in terms of metric gram units.

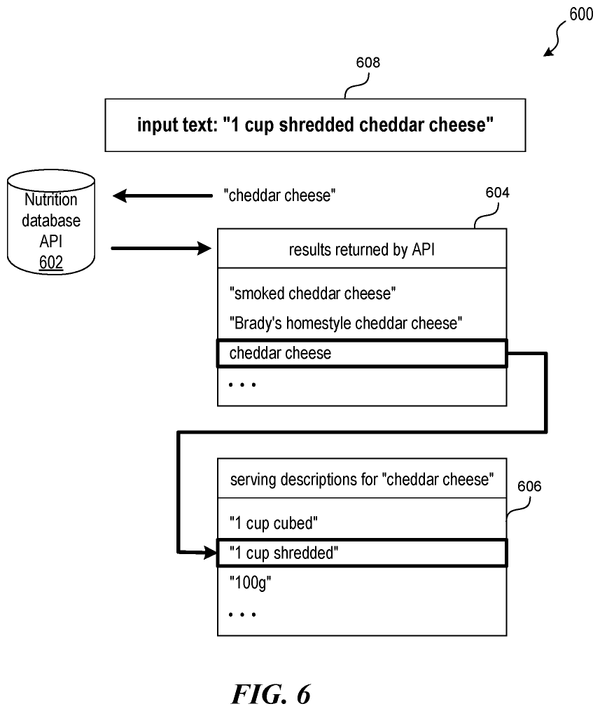

At step 208, the food logger system scales acquired nutrition information according to both the syntax of and explicit quantifiers given in a user's input text. The scaling and quantification module (SQM) can analyze and use information extracted by the CFLP and IAO and convert it to nutritional values specific to the user's described meal. The SQM can convert the information to nutritional values based on portion and quantity descriptors and interpretation rulesets 218. A nutrition database query can return multiple results from which the IAO evaluation module selects the most appropriate. Each individual result contains multiple nutrition information mappings, each with its own serving description which must subsequently be evaluated. FIG. 6 shows the difference between the results from the nutrition database and the serving descriptions associated with each result. The SQM is responsible for selecting the most appropriate serving description from the result chosen from the database query. The SQM bases this decision on the food item and portion identified by the CFLP module.

At step 210, the food logger system generates a structured output for both presentation to the user 212 and entry into the user's food log (e.g., dietary log 220). This module takes as input the scaled nutrition information from the SQM and generates as output both information to display to the user and data for ingestion into a food logging database. Information for display can be interface specific. Data intended for food logging can conform to the schema of the target database. The output module is configurable to interact with many user interfaces and database engines. Steps 202 and 210 are interactive digital systems and myriad embodiments of components for food logging tasks.

Steps 204, 206, and 208 can process and interpret user input to generate corresponding nutrition information constitute a unique and new system for logging dietary intake data. Steps 204, 206, and 208 can make use of the highly systematic and predictable patterns which English speakers often describe what they eat and drink at a meal. Within such patterns, words describing quantities or amounts consumed separate different components of a meal and typically precede the food entity they quantify. In addition, established usages of punctuation and conjunctions allow for unambiguous demarcation of individual components of a meal description, even if such a description is quite complex.

Example meal description: “I had two chicken cutlets with pasta, a side of sautéed broccoli, and a glass of wine for dinner.” Using the text that identify quantities (“two”, “a side”, and “a glass”) and the punctuation given to segment the description and ignoring words not associated with food items gives three distinct food entities (“chicken cutlets with pasta”, “sautéed broccoli”, and “wine”). Each of these entities can be associated with its respective preceding quantity. Parsing a meal description in this fashion eliminates many common but complex procedures associated with Natural Language Processing (NLP) such as part-of-speech tagging, lemmatization, and dependency tree construction with no loss of essential information.

As shown in FIG. 2, information enters the food logging system in the form of unstructured text via a Text Acquisition Module (TAM). This module constitutes one half of the user-facing portion of the food logging system, the other half being the Output Generation (OG) module that presents nutritional information calculated from input text back to the user 212. Text elicited from a user 212 by the TAM is passed first to the CFLP that carries out syntactic parsing and interpretation of the CFL expected from the user. Specifically, the CFLP exploits the properties of CFL to break up user input into pairs of individual food entities and their respective quantities. These food entity/quantity pairs are passed to the Information Acquisition Module (IAM) which queries external resources to obtain nutrition information for each food entity. The IAM employs a multi-step process for ensuring an optimal match for the information acquired and the food entity/quantity pairs provided by the CFLP. Part of this process can involve the IAM effectively asking the CFLP to further break down complex food-entities into smaller components more likely to be matched accurately to nutrition information. Thus, the IAM and CFLP can employ two-way interaction as the food logging system interprets user input. Once optimal nutrition information has been acquired by the IAM, both food entity/quantity pairs and this information are passed to the SQM which is ultimately responsible for adjusting the acquired information to agree with the food amounts indicated by user input.

FIG. 3 is a block diagram illustrating a process 300 for converting text into pairs of quantity and food terms, in accordance with embodiments of the present technology.

At step 302, the text standardization module can convert all text to a single case, removing stray or irrelevant words (“stopwords”) and punctuation, and ensuring no whitespace is either extraneous or missing. The text standardization module can convert the text according to text modification and substitution rulesets 310.

At step 304, the parsing module performs several text manipulations specific for the CFL expected as input. Specifically, indefinite articles “a” and “an” are converted to “one” as they represent quantifiers in the context of CFL, commas are replaced with “and”, and words considered immaterial are removed. These immaterial words consist of terms describing the particular preparation of a food entity that has no effect on its nutritional content, such as “chopped” or “broiled”. The parsing module can perform the text manipulations according to food term to nutrition information mapping sources and evaluation rulesets 314.

The standardized output described above is passed to the parsing module that performs several key tasks as follows: 1) a type is assigned to every word in the input, 2) single words are merged into multi-word entities where appropriate, and 3) the input is segmented into groups of words corresponding to a likely recognizable food entity and its associated quantifiers. As configured, the types assigned to each input word are derived from an external resource consisting of explicit lists of words. These lists consist of data types (lists, dictionaries, tuples) and are structured in a way that all important attributes of a given word can be determined by consulting these lists. For example, the word “steak” would be identified as and assigned the type of both a “main dish” and a “protein-forward” meal component while the word “cream” would be identified as a type of cheese, a beverage, and a recipe ingredient because it commonly occurs in all three of these contexts.

The word lists used by the CFLP were created through an empirical process of collecting and analyzing the following large datasets of nutrition information, recipes, ingredients, and restaurant menus (e.g., USDA “branded” and “foundation” foods).

The combined information from these data sources consists of unique food and food-related terms. This dataset can be reduced to a lower number of unique items through the following steps: 1) text standardization and de-duplication—dates and names can be removed; 2) entries containing more than more than two words but not containing any of the 1000 most frequent (2-4)-gram word pairs observed can be removed; 3) entries consisting of less than three characters can be removed; 4) entries containing more than 8 total words and any containing words observed less than 20 times across the corpus can be removed. Entries that contain misspellings and have no matching in the corpus but are common with minor spelling correction, are corrected to their corrected item; 5) entries containing 3 or more words can be grouped by similarity. Each group can be scored by observed word frequency and the highest scoring of each group can be taken as a representative while others may be removed. Similarity can be calculated as the degree of word set intersection. This step can select “milk chocolate cake” as the representative example for entries such as “hot chocolate cake” and “double chocolate cake”; and 6) all entries containing less than 3 words can be removed.

From this list of words and tabulated occurrences, the most common food words can be selected and used as the list of known food nouns for the CFLP. Several manual additions and deletions to this word list were made during further development. In addition, this word list was further segmented into specific categories of food and beverages (breads, pastas, proteins, drinks, spices, etc.).

Concurrently with the process noted above, a tally of food words separated by the conjunction “and” can be accumulated and the common instances where two food words were joined by “and” used to develop the ruleset whereby the CFLP can identify specific instances where “and” serves as an intrinsic part of a single food phrase (e.g., “hot and spicy”). Similarly, common 2-gram word pairs observed prior to step 2 described above can be used to encode a ruleset whereby such word pairs would be treated as a single food entity. The process of merging text fragments is iterative and convergent, thus several rounds of pairwise concatenation can occur, resulting in multi-word food phrases.

The word lists and rulesets created as above allow the CFLP to identify and demarcate food nouns and noun phrases. In order to develop a classifier to identify words that are likely or unlikely to accompany these food nouns and phrases, a word classifier can be built and trained as follows: 1) A corpus of 12k NPR articles can be passed through a pipeline that extracts all sentences that contained one or more food nouns; 2) the sentences can be stripped of known food nouns and the resulting stripped phrases were broken down into a unique set of words; 3) the same process can be done for the dataset used to derive CFLP word lists; and 4) the symmetric set difference of these two word sets can be used to calculate a score for any word in either set. Those words that occur only in the NPR corpus can have a negative value as these are assumed to be non-food related. Conversely, those that occur only within entries from the food noun source dataset can have a positive value as these are known to be food related. Score values were based on word frequency.

In some implementations, words most strongly score as being non-food related are forms of the verb “to be” (e.g., “are”, “was”, “were”) while the words most strongly identified as being food related included many adjectives commonly associated with foods (e.g. “balsamic”, “herb”, and “cajun”). This word-to-score mapping functions as a classifier that both predicts constituent words as being food- or not food-related and provides a score that reflects the confidence of such predictions. Within the CLFP and IAO, this classifier takes the form of a word-to-score mapping accessible to all modules.

In order to test the described classifier, a fraction (e.g., 10 percent) of all input data (food phrases from CFLP development and NPR sentences containing at least one known food noun) can be held out from the training process described. The classifier can be used to assign a score for each of these held out phrases by summing the individual word scores for all words known to the classifier. Phrases with a summed score greater than zero can be predicted to be food-related; those with negative scores can be predicted to be from NPR articles.

Similarly to lists of food nouns, the CFLP makes use of explicit lists of words and text to be considered as quantifier terms. Inspection of CFL revealed that such terms into 4 distinct categories: 1) numerical quantifiers as text or numerals (e.g. “two”, 5); 2) relative quantifiers (e.g. “large”, “medium”, “small”); 3) absolute portions corresponding to an established volume or mass (e.g. “cup”, “ounce”) or portions reasonably approximated by a fixed volume or mass (e.g. “bowl”, “glass”); and 4) self-referential portions or portions characteristically linked to a given food entity (e.g. “a slice” naturally pairs with “bread” or “pizza”, but not “soup”).

Word lists for the quantifier categories 1-3 can be compiled by extensive examination of CFL. In some cases, explicit lists for self-referencing or “natural” portions (4) were not assembled. Instead, the food logging system incorporates a separate mechanism for identifying such terms. This mechanism is embodied by different modules of the food logging system as described below.

In addition to word lists of foods and quantifiers, the CLFP makes use of a set of rules for interpreting input text. One set of rules directs how individual words or groups of words are interpreted while another ruleset directs how text is broken down into pairs of food entities and quantifiers.

At step 306, the food entity pattern matcher module employs a multi-step process for ensuring an optimal match for the information acquired and the food entity/quantity pairs provided by the CFLP. For word interpretation, the following rules can be implemented: 1) word-to-list matches are carried out so as to allow for matching singular to plural and vice versa; 2) words belonging to a multi-word food entity are appropriately demarcated. For example, “kidney” followed by “bean” would be considered as a single food entity “kidney bean” and not two distinct entities. Rules for making such word groupings are provided by an external ruleset (e.g., pattern matching rulesets 316) derived through a process similar to that used to generate the aforementioned word lists; 3) adjacent quantifier terms are treated as a single functional unit. Thus “two big bowls” is interpreted as a single quantifier term to be interpreted by the SQM; and 4) words that are food-related but that are not explicitly food nouns are identified by the word classifier described above.

The segmentation of text into individual food entities and quantifiers can be directed by the following rules: 1) the presence of a known food noun indicates the presence of a food entity; 2) quantifiers delimit the boundaries of a food-entity; 3) by default, “and” is treated as a delimiter that indicates that its preceding and following words belong to two distinct food entities. However, in some cases, the “and” conjunction occurs as an intrinsic part of a single food entity, such as “macaroni and cheese” or “pizza with pepperoni and olives”. In such cases, the “and” conjunction is not treated and an entity delimiter. The CFLP determines when not to use “and” as a delimiter by both consulting external lists of food nouns commonly joined by “and” and by analyzing syntactic patterns found within the text; and 4) quantifiers are inferred where appropriate. For example, “grapefruit and yoghurt” would be interpreted as “one grapefruit” and “one yoghurt”. In this fashion, all text between either two quantifiers or a single quantifier and the start or end of the meal description is treated as text corresponding to a single food entity.

Regarding rule 3, the CFLP can assign a role to “and” by identifying patterns of food nouns and conjoining words. This pattern matching takes the form of preserving common conjunctions while translating all food text into a token consisting of the text “FOOD/XXXX” where XXXX corresponds to the type of food the text describes (provided by the external word lists associated with the CLFP). For example, the phrase “chicken cutlets and linguini” would be tokenized to the pattern “FOOD/MAIN and FOOD/PASTA”. Assignment of such patterns to food entities serves two purposes. First, certain patterns can indicate that “and” should not be treated as a delimiter. Secondly, these patterns can direct the interpretation of individual food entities and quantifiers by the IAM. Consider the following example:

Raw User Input:
“I had a turkey sandwich with lettuce, tomato, and mayonnaise on wheat bread, a small bag of potato chips, and a diet coke for lunch”

Standardized User Input:
“I had one turkey sandwich with lettuce and tomato and mayonnaise on wheat bread and one small bag potato chips and one diet coke for lunch”

Food entities identified by the CFLP using quantifiers as delimiters:
#1: “turkey sandwich with lettuce and tomato and mayonnaise on wheat bread”
#2: “potato chips”
#3: “diet coke”

Corresponding Token Patterns:
#1: “FOOD/MAIN with FOOD/ZERO and FOOD/FRUIT and FOOD/SAUCE on FOOD/BREAD”
#2: “FOOD/MAIN”
#3: “FOOD/BEVERAGE”

In this example, the presence of pattern #1 indicates that the “and” conjunctions within the food entity should not be used as delimiters because they are serving to join words that describe a single complex, multi-part food entity. In addition to serving as a parsing directive, these token patterns can serve to guide how the IAM processes the strings containing the pattern. Details of pattern usage are described in the section on of the IAM. At step 308, the CFLP sends the paired portion/quantity terms and food entity phrases to the IAO component (FIG. 4).

FIG. 4 is a block diagram illustrating a process 400 for acquiring optimal nutrition information for each input, in accordance with embodiments of the present technology. The IAO component of the food logging system takes as input a single quantifier/food entity pair produced by the CFLP and generates as output a data object containing both this pair and one or more nutrition information mapping for the food entity selected as the most optimal from all made available from external resources. To accomplish this task, the IAO incorporates four distinct sub-components. At step 402, a resource query module acquires nutrition information mappings (e.g., from API to nutrition information mapping resource 412) that describe the amount of macronutrients in a given portion of a given food entity.

At step 404, a standardization module, such as a query result standardization module, converts these mappings to a standard format.

At step 406, a result evaluation module that both selects the most optimal nutrition information mapping from all those made available and determines if the input query needs to be re-parsed by the CLFP, at step 410. The result evaluation module can perform the selection and determination according to query/food entity matching and processing rulesets 414.

At step 408, a scaling directive module can use the pattern determined by the CLFP to adjust portion scales and amounts. The scaling directive module can adjust the scales and amounts according to the scaling rulesets 416. In addition, the result evaluation module examines portion and serving information acquired from external sources to both discard nutrition information mappings representing outliers and ensure that each and every query is paired with at least one nutrition information mapping defined in terms of metric gram units. The scaling directive module can send the query result with the nutrition information and scaling directives to the SQM.

The resource query module of the IAO consists of one or more API units, each of which can submit a query string to an external database, then returns a data object containing a nutrition information mapping along with both the portion/amount information and a string describing the entity to which the mapping applies.

Example Query Result:
Food description:
   “peanut butter”
Serving description:
   “two tablespoons”,
Nutrition information mapping:
   kcal=170,
   protein=22 g,
   etc.

Because not all external sources of nutrition information mappings conform to a single protocol for the data they return, the IAO incorporates a standardization module that transforms query results into a standard key/value mapping format similar to the structure of the example result above so as to be consistently interpreted by downstream modules.

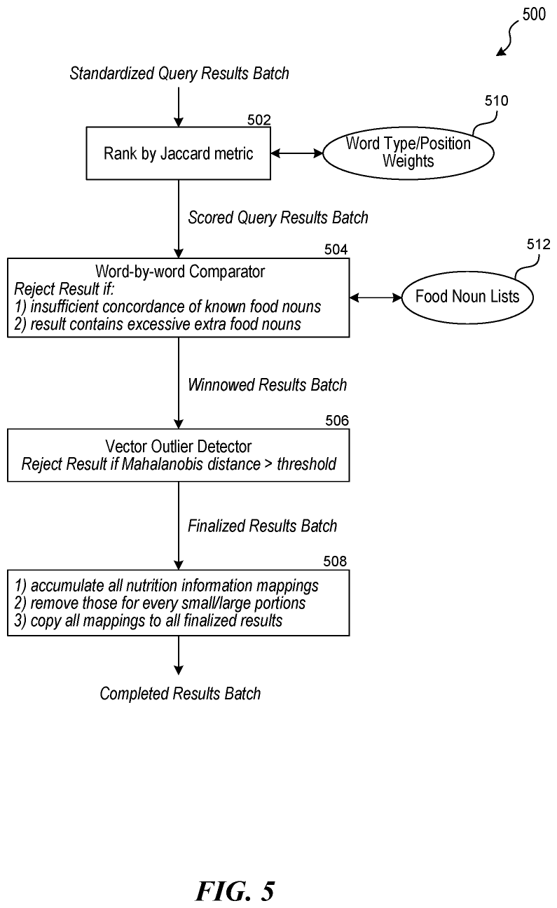

FIG. 5 is a block diagram illustrating a method 500 for culling and ranking nutrition information mappings, in accordance with embodiments of the present technology. Following standardization, query results are processed by the evaluation module of the IAO. Importantly, each externally configured resource can return an arbitrary number of query matches for a given string as these resources function essentially as search engines against a database with many candidate matches for the query. Moreover, each query result can contain multiple nutrition information mappings, each corresponding to a different portion size. It is the goal of the evaluation module to find the most relevant nutritional mappings from among results returned by all external resources. To perform this task, the module ranks, sorts, and, rejects entire queries or individual nutrition information mappings. The evaluation module will function with a single query result as input and will always output at least one result with a complete nutritional information mapping when provided with one or more results as input.

Example of query result rejected based on text properties:
Query:
   “pizza”
Result:
   Food description:
      “pizza with sausage, onions, and peppers”
Evaluation:
   Three food nouns in result not found in query
Action:
   Reject

In this example, the query text does not agree well with the food description text of the query result. Specifically, the result text contains many extra food nouns, indicating that the food described is not equivalent to that described by the query. Thus, the nutrition information accompanying this query result would lead to inaccurate calculations for total nutritional content of a meal and would be rejected by the IAM evaluation module.

Example of query results rejected based on numerical nutritional content:

Meal Description:
“two chicken cutlets with pasta, a side of sautéed broccoli, and a glass of wine”

CFLP Identified Food Entities:
1) “chicken cutlets with pasta”
2) “sautéed broccoli”
3) “wine”

Query results for “sautéed broccoli”:
1) 100 g of broccoli contains 35 kcal, 0 g fat, . . .
2) 1 cup sautéed broccoli contains 245 kcal, 7 g fat, . . .
3) 1 cup sautéed broccoli contains 256 kcal, 8 g fat, . . .
4) 1 side of sautéed broccoli contains 185 kcal, 6 g fat, . . .
5) 1 cup sautéed broccoli rabe contains 211 kcal, 9 g fat, . . .

In the example above, the CFLP extracts the query “sautéed broccoli” from the given meal description and the downstream query component returns the five results shown (nutrition information truncated). The results for “broccoli” (1) and “broccoli rabe” (5) should not be considered as sources of nutrition information because they describe food entities that differ substantially from that of “sautéed broccoli”. The food logging system removes such results from consideration via a combination of text comparison as above and nutrition information vectorization. In this case, result (1) does not contain the term “sautéed” which the food logging system considers to be a keyword when comparing queries and results. Similarly, result (5) would be rejected because it contains the term “rabe” not found in the query. The nutrition information vectorization methods of the food logging system convert the nutrition information within each result mapping to a normalized vector and these vectors are compared by a distance metric to identify outliers that should not be used as information sources for a given query. In the example results above, the result describing simply “broccoli” has significantly different fat content than the other four results, thus its vector representation would be quite removed from the ensemble average of all five results. Consequently, the evaluation module would exclude this result from further consideration.

The IAO evaluation module accomplishes this query result optimization via five distinct processing steps that cull a batch of input results into a batch results deemed acceptable for the original query. At step 502, the query string and the food description string of each result are compared on a word-for-word basis. For these comparisons, food and food-associated words known to the food logging system through its defined word lists are used for comparison. Each pairwise comparison is used to score the match according to the Jaccard metric (intersection-over-union) of the word sets of each respective string. Thus, each query result will receive a score from 0.0 to 1.0 inclusive with 1.0 indicating that all food nouns in the query were found in the result. The module also employs a modified version of this metric where words are weighted according to predefined significance and their position in a string. Queries with a score of 0.0, indicating no agreement between query and result, are rejected. The IAO evaluation module can assign weights to words according to the word type/position weights 510.

At step 504, the food description for each query result is evaluated against the original query string and results whose food descriptions contain too few of the known food words found in the original query are rejected. The evaluation process of the food descriptions can use food noun lists 512. Results whose descriptions contain an excessive number of extra known words are rejected similarly.

At step 506, nutrition information mappings from all non-rejected query results are vectorized using predefined transformations of specific macronutrient values. These vectors are normalized to allow comparison between different portion sizes. Then, the Mahalanobis distance of each vector to the ensemble centroid is calculated and those with a distance greater than a specific cutoff are rejected. This vector-based culling removes query results with markedly different ratios of macronutrients from the results batch as a whole. Thus, the food logging system is able to exclude results for simple “broccoli” when querying “sautéed broccoli”. This outlier analysis is performed when five or more nutrition information mappings are available.

At step 508, all nutrition information mappings at this point are interchangeable with respect to their ratios of macronutrients. Mappings corresponding to very small (“a grain of rice”) or very large (“a percolator of coffee”) portions are rejected by Z-score analysis. All accepted mappings are copied to all accepted query results.

Importantly, all results following step 506 have food descriptions with a high degree of textual similarity to the original query string and a high degree of agreement with respect to the nutrition information they contain. Thus, they can all be considered interchangeable. The evaluation module will always return at least one result. Should all results be rejected by the procedures above, the top scoring reject will be returned.

Equipped with these completed results, the IAO then decides if the best-scored of these results (by weighted Jaccard) is a suitable match for the original query. If so, this result is passed to the scaling directive module. If not, this string is passed back to the CFLP along with the directive to further break up the query into smaller food entities if possible. Should this fallback procedure fail, the top scoring result is passed to the scoring directive module regardless. Thus, the net effect of the IAO at this point is to find the most optimal query result for a given query string and to supplement this result with all applicable nutrition information mappings from all results of sufficient similarity. The evaluation module carries out an additional procedure at this point that ensures that the optimal query result returned contains at least one nutrition information mapping corresponding to metric units. Should a result not explicitly contain such a mapping, an existing mapping defined in units easily convertible to grams is scaled accordingly. Should no such mapping be available, a metric size is estimated from macronutrient content using an empirical linear model.

Lastly, the IAO employs a scaling directive module that annotates each query result with directives on how the portion for the associated food entity should be scaled. These directives are generated based on the pattern for the food entity generated by the CLFP.

Example of food entity re-parsed by the CLFP and reprocessed by the IAO
Original query:
   “a turkey sandwich with lettuce, tomato, and mayonnaise on wheat bread”
Pattern:
   “FOOD/MAIN with FOOD/ZERO and FOOD/FRUIT and FOOD/SAUCE on FOOD/BREAD”
Best result for original query:
   Food description:
      “turkey casserole with french bread croutons”
Evaluation:
   Insufficient agreement between query and result
Action:
   Re-parse by CLFP and re-query
New queries with pattern assignments:
   FOOD/MAIN=“turkey sandwich”
   FOOD/ZERO=“lettuce”
   FOOD/FRUIT=“tomato”
   FOOD/SAUCE=“mayonnaise”
   FOOD/BREAD=“wheat bread”
Scaling directives based on pattern rules:
   “turkey sandwich”=scale by 1.0 (main component)
   “lettuce”=scale by 0.0 (no nutritional content)
   “tomato”=assume 2 ounces (role as topping)
   “mayonnaise”=assume 1 tablespoon (role as topping)
   “wheat bread”=scale by 0.0 (redundant with sandwich)

As can be seen in the example above, the pattern identified by the CLFP is used by the IAO to generate scaling directives consistent with the role of and, therefore, the amount consumed of each component of the food entity. In effect, the combined action of the CLFP and IAO instructs the SQM to 1) ignore lettuce due to its negligible contribution to total nutritional content, 2) assign a portion to both tomato and mayonnaise consistent with their role as toppings, and 3) ignore “wheat bread” because it describes the sandwich and should not be considered an addition to the sandwich. Nutrition information mappings are not scaled directly at this point because the actual amount of a food entity consumed as indicated in the meal description depends not only on such scaling directives, but also on the explicit quantifiers within the meal description. Thus, the final scaling of nutrition information mappings is handled by the SQM component described below.

FIG. 6 is a diagram illustrating an example 600 for selecting an appropriate serving description from those available in the result chosen from the nutrition database, in accordance with embodiments of the present technology. The scaling and quantification module (SQM) is responsible for handling information extracted by the CFLP and IAO and converting it to nutritional values specific to the user's described meal. A nutrition database query returns multiple results from which the IAO evaluation module selects the most appropriate. Each individual result contains multiple nutrition information mappings, each with its own serving description which must subsequently be evaluated. FIG. 6 shows the difference between the results from the nutrition database and the serving descriptions associated with each result. The SQM is responsible for selecting the most appropriate serving description from the result chosen from the database query. The SQM bases this decision on the food item and portion identified by the CFLP module.

Two separate decision-making processes have been designed, the first of which is known as the “Eigenportion” selection method. The term Eigenportion can refer to a serving description that is highly similar to the user inputted text. An Eigenportion can reflect inherent portions that are associated with food items, for example ‘slice’ goes with the food items ‘pizza’, ‘cake’ and ‘bread’. This similarity metric takes advantage of a user inputting a contextually appropriate portion word that is likely to be found in the set of serving descriptions associated with the chosen database result.

The set of words in the combined food and portion terms is compared with the set of words in each serving description. Jaccard similarity is used as the similarity metric (i.e. intersection-over-union). Note that word weights are not used in the calculation of this similarity score as is sometimes the case in the IAO module. A threshold of 0.2 is used to determine whether the input food text is an Eigenportion. The highest scoring serving description is selected in cases where multiple descriptions surpass the Eigenportion score threshold. Example 600 of FIG. 6 illustrates the input text 608 of “1 cup shredded cheddar cheese” in which “cheddar cheese” is input in the nutrition database API 602. The nutrition database API 602 returns results 604 with cheddar cheese selected and serving descriptions 606 with “1 cup shredded” selected.

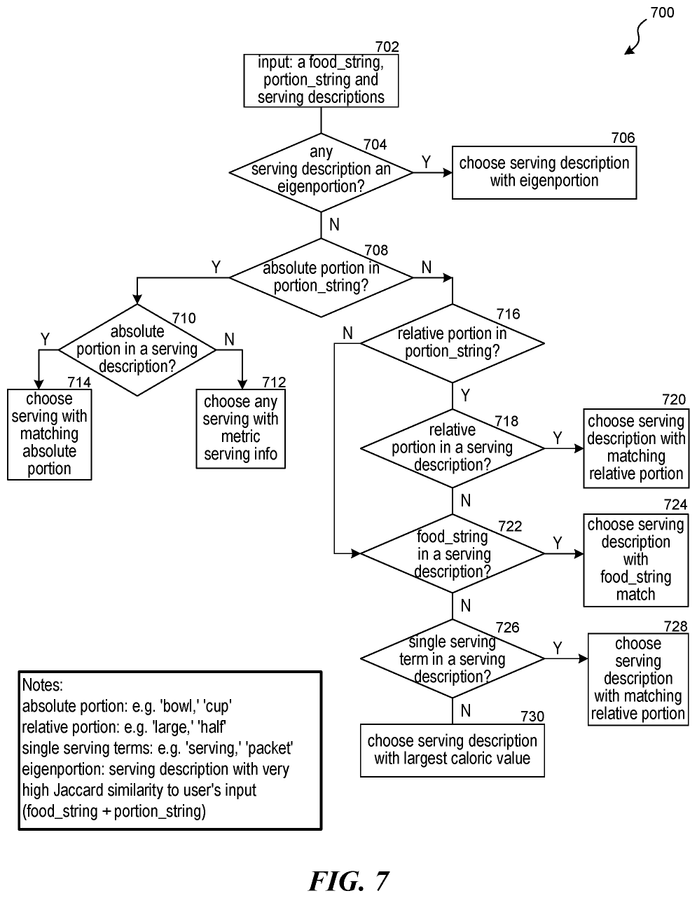

FIG. 7 is a block diagram illustrating a process 700 for selecting a serving description, in accordance with embodiments of the present technology. The second process for selecting the most appropriate serving description is called the “waterfall” decision method. It is invoked if the Eigenportion decision process outlined above fails to classify any serving description as an Eigenportion. The waterfall decision method consists of a set of decision rules which when followed in sequence identify the best serving description for a set of serving descriptions which do not qualify as Eigenportions.

At step 702, process 700 receives an input (a food_string, portion_string and serving descriptions). At step 704, process 700 determines whether any serving description is an Eigenportion. If a serving description is an Eigenportion, at step 706, process 700 choses a serving description with an Eigenportion.

If a serving description is not an Eigenportion, at step 708, process 700 determines whether an absolute portion term is found in the portion words (portion string) identified by the CLFP. If an absolute portion term is found, at step 710 process 700 determines whether an absolute portion term is found in the serving description. If an absolute portion term is found in the serving description, at step 714 process 700 selects a serving description which includes the same absolute portion term. If no serving description contains the absolute portion term, at step 712 process 700 selects any serving description which includes its serving weight information (in grams). The serving weight is subsequently used to calculate the absolute portion weight using portion weights defined by the food logging system.

If an absolute portion term is found, at step 716 process 700 determines whether there is a relative portion in the portion string. If a relative portion is found in the portion string, at block 718 process 700 determines whether there is a relative portion in the serving description. If a relative portion is identified by the CLFP and an available serving description contains that same relative portion term, at step 720 process 700 selects that serving description with the matching relative portion. In the case of multiple matches, process 700 selects the first such serving description identified.

If a relative portion is not found in the portion string or a relative portion is not found in the serving description, at step 722 process 700 determines whether there is a food string in the serving description. If an available serving description contains the food term identified by the CLFP, at step 724 process 700 selects that serving description with the food string match. In the case of multiple, process 700 selects the first such serving description identified.

If there is not a food string in a serving description, at step 726 process 700 determines whether there is a single serving term in a serving description. If an available serving description contains a single serving term, at step 728 process 700 selects the serving description with a matching relative portion, such as with the highest priority term. The priority order of single serving terms in order from highest to lowest is as follows: “serving”, “portion”, “package”, “sandwich”, “cookie”, “cracker”, “cup”. In the case of multiple serving descriptions at the same priority level, select the first such serving description identified. If process 700 fails to select a serving description based on the decision sequence above, at step 730 process 700 selects the serving description with the highest caloric value.

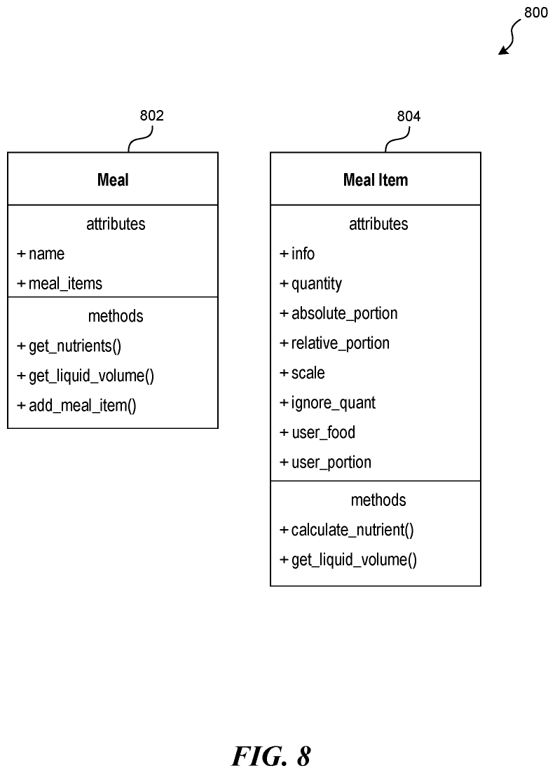

FIG. 8 is a diagram illustrating an example 800 for storing information related to a food logging instance, in accordance with embodiments of the present technology. The SQM stores two types of objects. The first of which represents the characteristics and portion information of individual food items extracted by the CFLP and IAO. These objects are referred to as ‘Meal Items’. The second object represents the entire meal as a whole, in the sense that a meal is made up of multiple food items. These are referred to as ‘Meal’ objects. FIG. 8 illustrates the UML diagrams for these two classes of Meal 802 and Meal Item 804. The SQM stores data related to each meal in a hierarchical fashion. Meal Item objects store the information extracted for each food item extracted from the user's text and a Meal object stores a list of Meal Item objects.

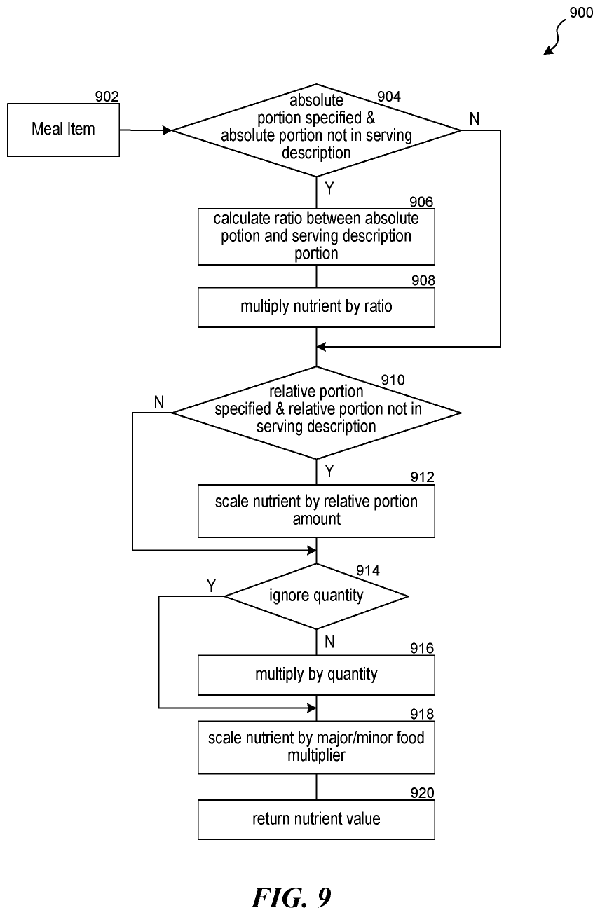

FIG. 9 is a block diagram illustrating a process 900 for calculating a nutrient value given the information extracted by the CFLP, in accordance with embodiments of the present technology. The Meal Item class contains methods for calculating nutrition information based on the food identified and portion stated. Total values for individual nutrients are calculated using the initial values received from the database query and then are scaled appropriately based on the portion information stored in the Meal Item class. At step 902, process 900 (e.g., nutrient scaling process) retrieves the Meal Item information.

At step 904, process 900 determines whether an absolute portion is specified by the user and the absolute portion term not found in any of the chosen food item's serving descriptions. If an absolute portion is not specified by the user and the absolute portion term is not found in any of the chosen food item's serving descriptions, process 900 proceeds to step 910.

If an absolute portion is specified by the user and the absolute portion term is not found in any of the chosen food item's serving descriptions, at step 906 process 900 calculates the ratio between the absolute portion weight in grams and the metric serving weight also in grams. At step 908, process 900 multiplies the nutrient amount by the above ratio.

At step 910, process 900 determines whether a relative portion is specified by the user and the relative portion term is not found in any of the chosen food item's serving descriptions. If a relative portion is specified by the user and the relative portion term is not found in any of the chosen food item's serving descriptions, at step 912 process 900 multiplies the nutrient amount by the relative portion's respective multiplier (e.g., “large” corresponds to a multiplier of 1.5).

If a relative portion is not specified by the user and the relative portion term is not found in any of the chosen food item's serving descriptions or after the nutrient amount is scaled (at step 912), at step 914 process 900 determines whether directives passed by the IAO request that quantity is be ignored. If directives passed by the IAO request that quantity is not to be ignored, at step 916 process 900 multiplies the nutrient amount by the quantity identified by the IAO module.

At step 918, process 900 multiplies the nutrient amount by a scaling factor based on the food item's status as either a major or minor food item. This status is determined by the IAO module. Major food items' nutrient information is left alone, i.e., scaled by 1.0. Minor and very minor food items' nutrient information are scaled by 0.5 and 0.1 respectively. At step 920, process 900 returns the nutrient value.

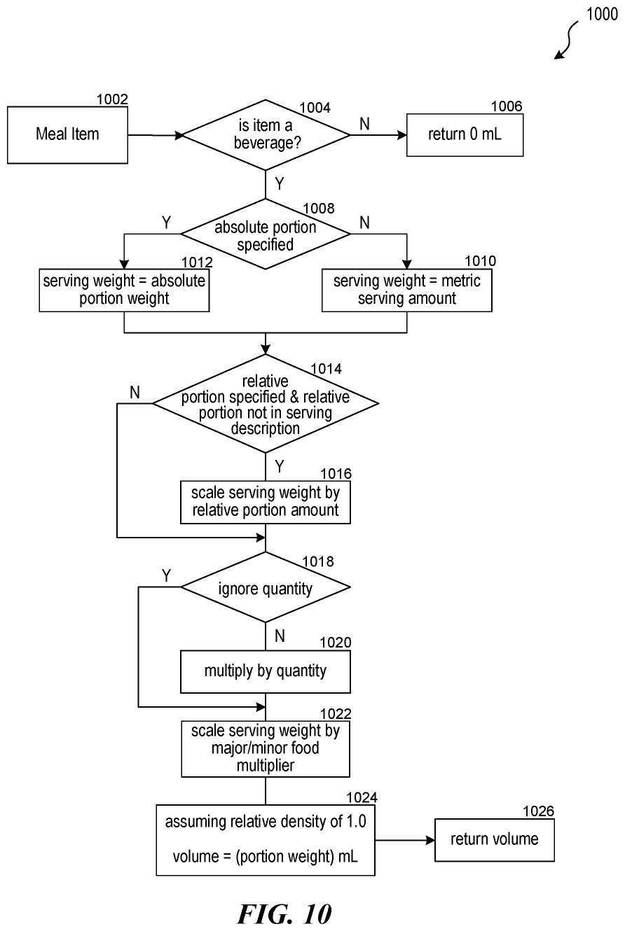

FIG. 10 is a block diagram illustrating a process 1000 for calculating liquid volume for a food item given the information extracted by the CFLP, in accordance with embodiments of the present technology. In addition to the values provided by the scaling of nutritional information provided by the IAO, the SQM is able to calculate the total volume of liquid contained in a meal by examining which Meal Item instances consist of beverages. The current list is not exhaustive and can be expanded to include more beverage types if required.

At step 1002, process 1000 (e.g., volume calculation process using a Meal Item object) retrieves the Meal Item information. At step 1004, process 1000 determines whether the food item is identified as a beverage. If the food item is not identified as a beverage by the food logging system, at step 1006 process 1000 returns 0 mL as the liquid volume.

If the food item is identified as a beverage, at step 1008 process 1000 determines whether an absolute portion is specified by the user. If an absolute portion is specified by the user (e.g. “bowl”, “glass”), at step 1012 process 1000 takes the serving weight to be the weight assigned to that portion term by the food logging system.

If no absolute portion is specified, at step 1010 process 1000, takes the serving weight to be the serving amount in grams returned by the nutrition database for the given food item. At step 1014, process 1000 determines whether a relative portion is specified by the user and the relative portion term is not found in the serving description.

If a relative portion is specified by the user and the relative portion term is not found in the serving description, at step 1016 process 1000 scales the serving weight by the relative portion's respective multiplier (e.g., “large” corresponds to a multiplier of 1.5).

At step 1018, process 1000 determines whether to ignore the quantity. If directives passed by the IAO request that quantity is not to be ignored, at step 1020 process 1000 multiplies the serving weight by the quantity identified by the IAO module. At step 1022, process 1000 multiplies the serving weight by a scaling factor based on the beverage's status as either a major or minor food item.

At step 1024, assuming all liquids have a relative density of 1.0, process 1000 takes the liquid volume as the serving weight in mL. At step 1026, process 1000 returns the volume. The output module takes as input the scaled nutrition information from the SQM and generates as output both information to display to the user and data to ingest into a food logging database. Information for display is interface specific. Data intended for food logging can conform to the schema of the target database. Regardless, the output module is configurable to interact with many user interfaces and database engines.

For each food description, nutrition information in the form of total Calories (kcal), protein (g), total fat (g), and total carbohydrate (g) can be associated by using resources such as USDA, NutritionX, and FatSecret. This annotation process can replicate what a practitioner of dietary logging would perform manually. That is, to look up a food item consumed, make a reasonable approximation of portion size, adjust nutrition information accordingly. For example, for a first example of “One slice of whole wheat bread”, a typical logger could search for “nutrition for one slice of whole wheat bread”. The returned result from this search query can show values for the macronutrients noted above (kcal=69, fat=0.9 g, etc.) and the source of the nutrition data (USDA). For a more complicated meal, an annotator would have to perform a similar search for each component and the information for each component to be in-line with the meal description. For example, a search for nutrition information for “blueberries” returns a USDA derived listing of values for 1 cup of blueberries. Therefore, an annotator would have to make a reasonable estimate of what portion of a standard cup “one handful” represents and scale the nutrition information results accordingly. Consequently, annotated values for macronutrients should be considered reasonable best guesses consistent with variations in human estimation. It is the goal of the food logging system to at least replicate the accuracy of this estimation, if not improve upon it.

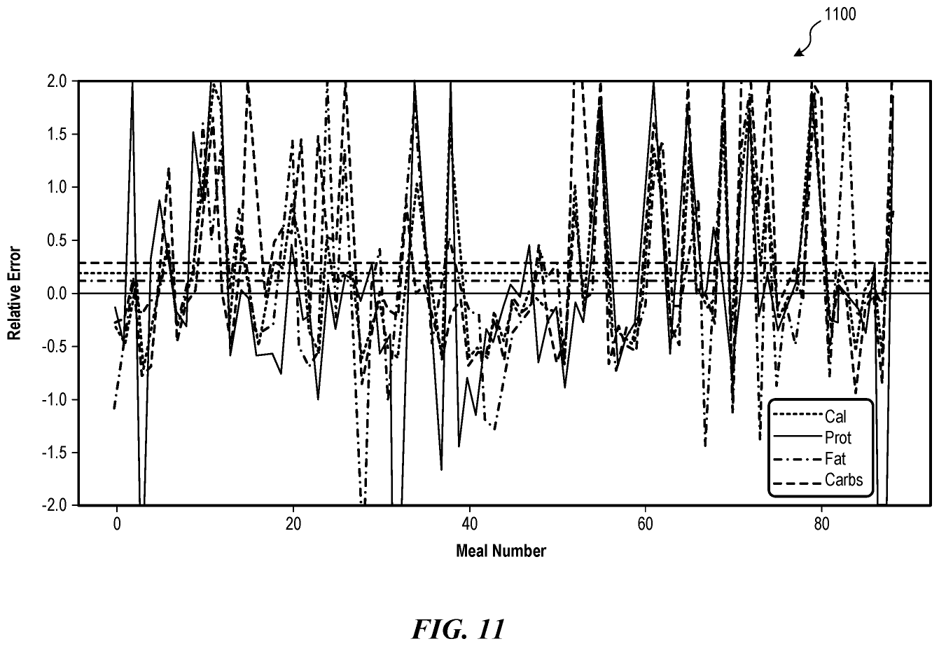

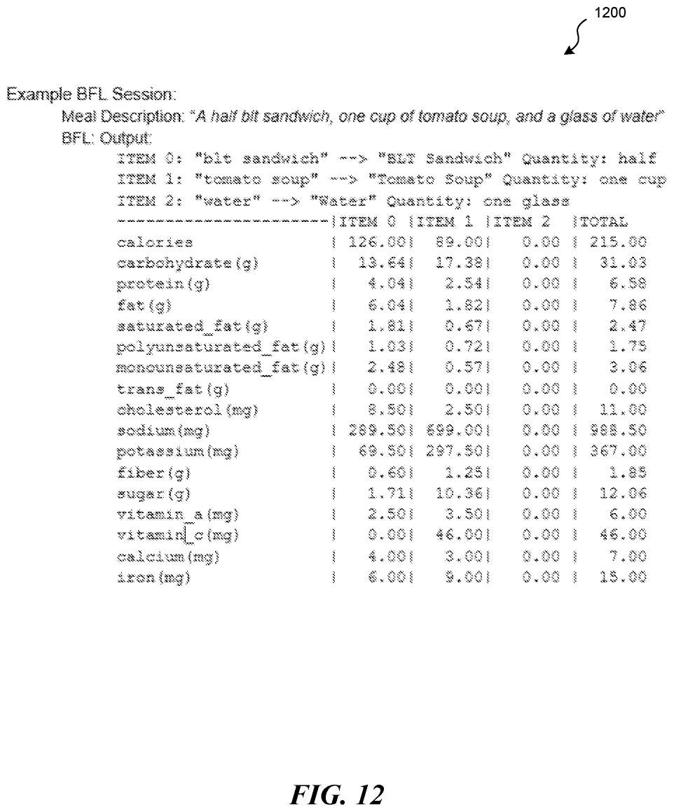

In an example, 88 annotated meals were run through the entire food logging system workflow and tabulated values for the four noted macronutrients output by the Output Module were recorded. The relative error for each macronutrient was averaged across all meals. An example food logging system session is given below with both meal description, food logging system output, and error summary. FIG. 11 shows a plot of relative macronutrient errors for each meal and results are summarized in Table 1 and Table 2. For all calculations, relative error was defined as: Relative Error=(annotated_value−food logging system_value)/annotated_value. FIG. 12 illustrates example 1200 of a food logging session.

TABLE 1

| | | Annotated | |
|---|---|---|---|
| Macronutrient | BFL Estimate | Value | Error |
| Calories (kcal) | 215 | 309 | 30% |
| Total Fat (g) | 7.9 | 14.6 | 46% |
| Protein (g) | 6.6 | 11.8 | 44% |
| Carbohydrates (g) | 31 | 32 | 3% |

| | |
|---|---|
| Average Error | 31% |

FIG. 11 illustrates graph 1100 illustrating food logging system accuracy for annotated meals. Annotated meals by number given on the X-axis and absolute relative error given on the Y-axis. Solid lines correspond to errors for individual macronutrients. Horizontal dashed lines are global averages for each macronutrient error. (Note: error values were capped at 200% to remove outliers)

TABLE 2

| Macronutrient | Average Relative Error (%) |
|---|---|
| Calories (kcal) | 17.7% |
| Protein (g) | 0.0% |
| Total Fat (g) | 11.0% |
| Carbohydrates (g) | 27.6% |

Food logging system nutrition contents estimates often have errors of 50% or more. However, the analysis described above relies upon human annotation of the meal descriptions themselves—a process subject to significant variability itself. That is to say that the values to which food logging system estimates themselves are compared should not be taken as absolute truths. In fact, two different annotators can arrive at very different results for the same meal description. Moreover, a food logging user will inevitably make estimates of the portions they consumed, and these estimates will certainly differ from true values. Half of the 88 nutritional summaries returned by the food logging system for this testing set evinced errors of 50% or less.

Analysis of the results above can lead to the following observations: 1) the nutrient estimates of the food logging system are more accurate when meal descriptions contain food amounts with precise quantities such as “grams” or “cups”; 2) nutrient estimates are more accurate for syntactically simple meal descriptions. Such meal descriptions can contain many items, but the less complex each item is, the better the food logging system is able to process the description and identify which items should be considered as meal components and which are parts of more complex, multi-part items. In the examples above, the “turkey sandwich with lettuce, tomatoes, and mayonnaise on wheat bread” is more difficult for the food logging system to interpret than “turkey sandwich with mayonnaise”, even though both describe essentially the same thing; 3) the more conforming a user's input is to CFL, the better able the food logging system is to correctly segment and process the input. This observation comes as no surprise because the entire CFLP has been designed around the expectation of receiving CFL as input. Variants with which the food logging system struggles might include putting quantities after their respective meal items (e.g. “baked potato [one], green beans [½ cup]”) or less common food phrase constructions such as “anchovies, onions, and garlic on pizza”; 4) the food logging system has difficulty with certain food items that can be quantified both in terms of individual units or a bulk quantity. Examples include “mixed nuts” or “raisins”. A phrase such as “20 raisins” may be mistakenly interpreted by the food logging system as “20 servings of raisins”, leading to a gross overestimate of the amount consumed; 5) accuracy of the food logging system is highly dependent on the accuracy of the underlying nutrition information mapping sources queried by the IAO. Certain resources, particularly FatSecret have erroneous values with respect to portion size. Also, as configured, the food logging system will pick randomly from query results deemed of equivalent quality. For example, in one session, the food logging system may choose a query result for “Thai vegetable stir-fry” for a query of “veggie stir-fry” while another session may select “gluten-free vegetable stir-fry” as these two results match equally well with the query with respect to the words the food logging system uses when comparing queries and results. Thus, there is run-to-run variation in food logging system estimates; 6) nutrient estimates are liable to be underestimated when the consumption of a whole food item is implied but a serving description representing the whole item is not available. In cases where gram amounts or absolute portions are not stated by the user, serving descriptions with single serving terms are likely to be selected. This selection can lead to an underestimation of nutritional values especially if a typical serving of a given food item is less than its whole. An example of such a case is an implied whole “avocado” where the selected serving description refers to a quarter of a whole avocado; 7) user expectations are highly variable and don't always align with how the food logging system is engineered to log food. The food logging system, and the IAO query evaluation module can place more weight on the most important words in a food string. This inherently means less weight on adjectives. However, regarding food, adjectives can change the NI of the foods described entirely. An example of this in action is “vegan sugar free banana bread” being matched with a result for “banana bread”. As per food logging system's design, this matching is a clear success as the CFLP and IAO modules identified the most important words, i.e. “banana” and “bread” to constitute the core of the query. However a user might object on the basis that standard banana bread has a different nutrition profile than a vegan, sugar-free version; and 8) users are liable to expect the food logging system to infer unstated information from their meal descriptions. It is true that nutritional information for foods can differ dramatically depending on the implied context. An example is the input string “turkey with whole wheat bread, lettuce and tomato”. Taken on its own the “turkey” in this query will likely match with plain home-cooked style turkey meat. Taken in the context of a sandwich, which this input string was intended to represent, the “turkey” should instead match with sliced deli-style turkey meat which has a far higher sodium content than home-cooked turkey meat.

Examples of Good Food Logging System Results:
1) Meal description:
   “a roast beef sandwich, a bottle of beer, and two pickles”
   Errors:
   Calories: 15%
   Fat: 38%
   Protein: 6%
   Carbohydrates: 11%
   Average: 18%
Summary:
   A three-part, syntactically simple meal description easily interpreted by the food logging system. The error values observed seem entirely consistent with variations in sizes and macronutrient ratios among sandwiches and beer.
2) Meal description:
   “two breakfast tacos with sour cream and salsa”
   Errors:
   Calories: 17%
   Fat: 25%
   Protein: 19%
   Carbohydrates: 8%
   Total: 17%
Summary:
   A single-part meal description containing a main item (breakfast tacos) with accompaniments (“sour cream”, “salsa”). The food logging system added nutritional information for the accompaniments in proper proportions, leading to quality estimates of overall nutritional content.

Examples of Sub-Optimal Food Logging System Results:
1) Meal description:
   “black bean soup, asparagus, salad, sweet potato, 2 glasses red wine”
   Errors:
   Calories: 6%
   Fat: 1372%
   Protein: 109%
   Carbohydrates: 8%
   Total: 373%
   Summary:
   A multi-part meal description for which two macronutrient values are grossly misestimated. These misestimates stem from two underlying errors. Firstly, the nutritional information acquired by the food logging system estimated the fat content of “black bean soup” to be 10 g per serving, while the annotator estimate was 1.5 g. Secondly, the lack of quantifier terms in the description caused the food logging system to misinterpret the soup as both a main component and as a topping for the sweet potato. Thus, the nutritional content contributed by the soup was included twice in the final tally, further inflating the already overestimated fat content of the meal.
2) Meal description:
   “Two fried eggs with half a slice of pumpernickel toast and half a chapati, and one smoothie with half a glass of milk, one scoop of vanilla whey powder, one banana, a handful of blueberries”
Errors:
   Calories: 83%
   Fat: 110%
   Protein: 61%
   Carbohydrates: 55%
   Total: 77%
Summary:
   Despite the complexity of this meal description, the food logging system correctly identified 7 out of 8 components and their associated quantities and acquired reasonable nutrition information for each. The errors for this example arise from 1) discrepancy between annotator values and food logging system estimates for “fried eggs” and “whey powder” and 2) the failure of the food logging system to recognize the components “milk”, “whey powder”, “banana”, and “blueberries” as ingredients of the smoothie. Instead, the food logging system not only included these ingredients individually, but also included nutritional content of a “smoothie” as a distinct food item, leading to large overestimation of nutritional content.

The results and examples above highlight both the capabilities and limitations of the current implementation of the food logging system. Most importantly, the food logging system is able to accomplish its primary goal. Namely, to provide a means of translating unstructured user input into reasonably accurate nutrition information. The food logging system is able to process complex meal descriptions and, while the accuracy of its results improves with the more detailed and highly structured the user input, the food logging system is still able to provide reasonable results for a very wide range of input cases.

The input and output modules (TAM and OG) are easily configurable to connect to myriad different UI interfaces as well as database systems. Likewise, the IAO can be easily configured to connect to any additional or helpful nutrition information mapping resources deemed necessary. Thus, the current state of the food logging system can be fairly described as a “minimum viable product” that accomplishes its goals and allows for easy testing, demonstration, and augmentation of its capabilities.

Additional development can include: 1) addition of a “sanity check” mechanism that flags and possibly modifies extreme and likely erroneous nutrition information estimates. This addition would ensure that errors such as interpreting “10 mixed nuts” as 10 servings of mixed nuts are avoided; 2) addition of a further layer of user interaction that allows a user either to confirm results returned by the food logging system or to provide a means of adjusting values as necessary. Keeping in the spirit of the food logging system, such user interventions should be simple and straightforward such as allowing a user to simply scale returned values to match their portion estimates (e.g., a slider bar) or to clarify parts of their input should they find the results of the food logging system to be inconsistent with their input. For example, if the food logging system returns a result for “chocolate ice cream” for a query of “a cookies and cream chocolate bar”, the food logging system might provide a means of entering different or alternative descriptions of this item to allow for a better match; 3) further augmentation of the pattern matching procedures of the CFLP to allow for better processing of more complex meal items; 4) the inclusion of a clear prompt or instructions about how best to enter meal descriptions. Rather than a detailed description of what type of inputs the food logging system works best with, a simple prompt in the form of what such language would look like may suffice. Example prompt: Please describe your meal including all food and beverages along with amounts consumed. You can say or type something like “two scrambled eggs, a piece of toast, a small bowl of mixed fruit, and a cup of coffee”. This prompt cues the user to use the syntax most likely to be correctly processed by the food logging system. 5) a further round of code refactoring to make the correlations between the modules as described above consistent with the actual structure of the source code files; and 6) addition of a caching mechanism to avoid re-querying the same food items repeatedly.

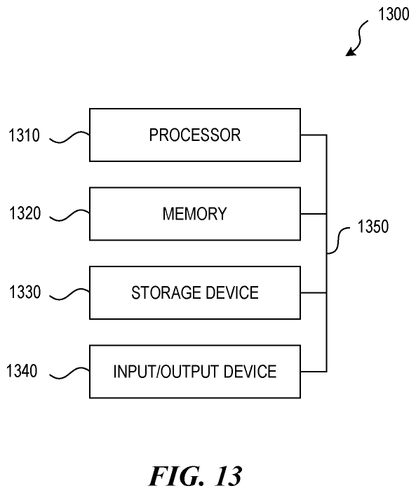

FIG. 13 is a schematic block diagram of a computing system or device (“system 1300”) configured in accordance with embodiments of the present technology. The system 1300 can be incorporated into or used with any of the systems and devices described herein, such as the system 100 and/or user devices 104 or analyzing devices 102 of FIG. 1. The system 1300 can be used to perform any of the processes or methods described herein with respect to FIGS. 1-10. The system 1300 can include a processor 1310, a memory 1320, a storage device 1330, and an input/output device 1340. Each of the components 1310, 1320, 1330 and 1340 can be interconnected using a system bus 1350. The processor 1310 can be configured to process instructions for execution within the system 1300. In some embodiments, the processor 1310 can be a single-threaded processor. In alternative embodiments, the processor 1310 can be a multi-threaded processor. Although FIG. 13 illustrates a single processor 1310, in other embodiments the system 1300 can include multiple processors 1310. In such embodiments, some or all of the processors 1310 can be situated at different locations. For example, a first processor can be located in a sensor device, a second processor can be located in a user device (e.g., a mobile device), and/or a third processor can be part of a cloud computing system or device.

The processor 1310 can be further configured to process instructions stored in the memory 1320 or on the storage device 1330, including receiving or sending information through the input/output device 1340. The memory 1320 can store information within the system 1300. In some embodiments, the memory 1320 can be a computer-readable medium. In alternative embodiments, the memory 1320 can be a volatile memory unit. In yet some embodiments, the memory 1320 can be a non-volatile memory unit. The storage device 1330 can be capable of providing mass storage for the system 1300. In some embodiments, the storage device 1330 can be a computer-readable medium. In alternative embodiments, the storage device 1330 can be a floppy disk device, a hard disk device, an optical disk device, a tape device, non-volatile solid-state memory, or any other type of storage device. The input/output device 1340 can be configured to provide input/output operations for the system 1300. In some embodiments, the input/output device 1340 can include a keyboard and/or pointing device. In alternative embodiments, the input/output device 1340 can include a display unit for displaying graphical user interfaces.

Non-transitory computer program products (i.e., physically embodied computer program products) are also described that store instructions that, when executed by one or more data processors of one or more computing systems, cause at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The systems and methods disclosed herein can be embodied in various forms including, for example, a data processor, such as a computer that also includes a database, digital electronic circuitry, firmware, software, or in combinations of them. Moreover, the above-noted features and other aspects and principles of the present disclosed implementations can be implemented in various environments. Such environments and related applications can be specially constructed for performing the various processes and operations according to the disclosed implementations or they can include a general-purpose computer or computing platform selectively activated or reconfigured by code to provide the necessary functionality. The processes disclosed herein are not inherently related to any particular computer, network, architecture, environment, or other apparatus, and can be implemented by a suitable combination of hardware, software, and/or firmware. For example, various general-purpose machines can be used with programs written in accordance with teachings of the disclosed implementations, or it can be more convenient to construct a specialized apparatus or system to perform the required methods and techniques.

The systems and methods disclosed herein can be implemented as a computer program product, i.e., a computer program tangibly embodied in an information carrier, e.g., in a machine readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

These computer programs, which can also be referred to programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user can provide input to the computer. Alternatively or in combination, the display device can be a touchscreen or other user input device configured to accept tactile input (e.g., via a virtual keyboard and mouse). Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input.

The technology described herein can be implemented in a computing system that includes a back-end component, such as for example one or more data servers, or that includes a middleware component, such as for example one or more application servers, or that includes a front-end component, such as for example one or more client computers having a graphical user interface or a Web browser through which a user can interact with an implementation of the subject matter described herein, or any combination of such back-end, middleware, or front-end components. The components of the system can be interconnected by any form or medium of digital data communication, such as for example a communication network. Examples of communication networks include, but are not limited to, a local area network (“LAN”), a wide area network (“WAN”), and the Internet.

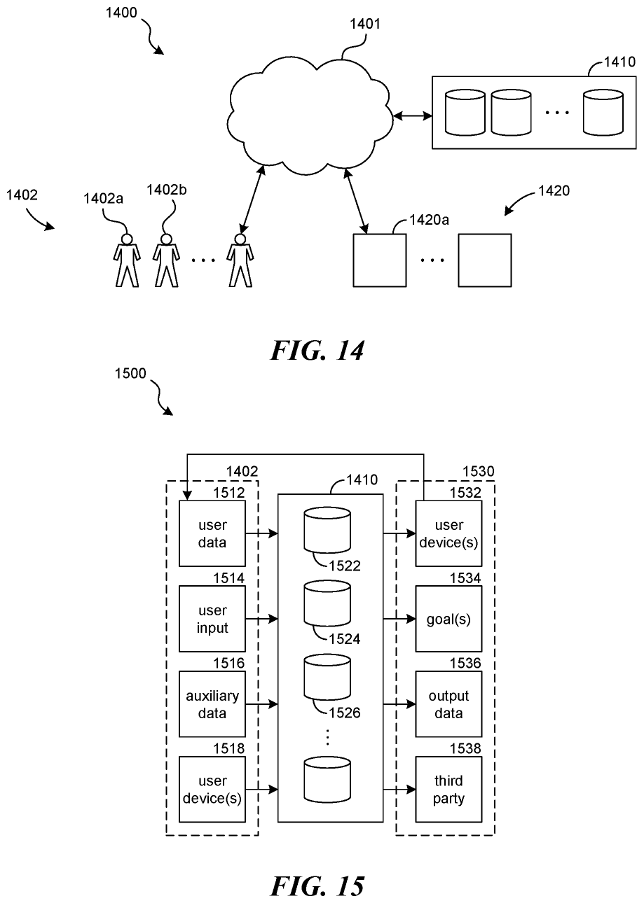

The computing system can include clients and servers. A client and server are generally, but not exclusively, remote from each other and typically interact through a communication network. The relationship of client and server arises by virtue of computer programs running on the respective computers and having a client-server relationship to each other. The computing system can be part of or incorporated into the systems discussed in connection with FIGS. 14 and 15.

FIGS. 14 and 15 are schematic diagrams illustrating exemplary computing environments in which a healthcare guidance system operates, in accordance with embodiments of the present technology. A system 1400 can include a network 1401, a biomonitoring and healthcare guidance system 1410 (“system 1410”), users or user devices 1402 (“user devices 1402”), and additional systems 1420. The network 1401 can transmit data between the user devices 1402, healthcare guidance system 1410, and/or additional systems 1420. The system 1410 can include analyzing devices, select one or more databases, models, and/or engines to analyze received data to provide self-care modes, predictions, identify contributing health factors, or the like. The description of system 100 of FIG. 1 applies equally to the system 1400 unless indicated otherwise, and the system 1400 can perform the methods disclosed herein.

The system 1410 can include databases, models, systems, and other features disclosed herein and can include models, algorithms, engines, features, and systems disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; PCT App. No. PCT/2021/028445, and other patents and applications discussed herein. For example, the system 1410 can receive health data (e.g., glucose levels, blood pressure, etc.) from user devices disclosed in U.S. application Ser. No. 16/888,105 or U.S. application Ser. No. 17/236,753 and can forecast or predict one or more health metrics disclosed in U.S. application Ser. No. 16/888,105 or U.S. application Ser. No. 17/167,795. The self-care modes can be periodically or continuously generated based on predicted or detected events, user settings, schedules, or the like. Forecasted metrics can be used to determine a behavioral intervention plan, turn-by-turn health plan, etc. In some implementations, the system can receive information about a predicted event, such as a hypoglycemic or hyperglycemic event. The system can identify the event and then periodically (e.g., hourly, daily, weekly, monthly) or continuously (e.g., in response to continuously received CGM data) recommend self-care modes to reduce or avoid the risk of predicted event.

The system 1400 can provide healthcare support in the form of behavioral interventions to achieve exercise goals. For example, the user 1402b can be training to increase cardiovascular health. The system 1400 can receive user exercise data (e.g., workout type, workout duration, etc.), exercise data (e.g., heart rate, blood pressure, etc.), positioning data (e.g., GPS data), or other data. The healthcare guidance system 1410 can use the data (all the data or subset of the data) determine healthcare support actions and behavioral interactions to be performed to, for example, develop behavioral intervention plan for completing workouts. The healthcare guidance system 1410 can use forecasting models or engines to determine recommendations for the user and can generate new models based on newly available data. The forecasting models or engines can be used for multiple users or a single user. In some embodiments, data associated from a user can be inputted into different models or engines and the output from those engines or models can be grouped, processed, and/or feed into additional models or engines, including those disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; and PCT App. No. PCT/2021/028445. For example, the healthcare guidance system 1410 can perform one or more steps of the method 200 of FIG. 2 or method 900 of FIG. 9 using output or data from patents and applications referenced herein.

The network 1401 can communicate with an auxiliary computing system 1420 that can provide programs, or other information used to manage the collection of data. For example, a computing system 1420a can communicate with a wearable user device 1420a to provide firmware updates. The healthcare guidance system 1410 can automatically update databases, models, and/or engines based on changes to the user device 1402a based on the update. The computing system 1422a and healthcare guidance system 1410 can communicate with one another to further refine data analysis.

A user can manage privacy and data settings to control data flow. In some embodiments, one of the computing systems 1420 is managed by the user's healthcare provider so that received user data is automatically sent to the user's physician. This allows the physician to monitor the health and progress of the user. The physician can be notified of changes (e.g., health-related events) to provide further reinforcement monitoring, new health goals, history data, modified health metrics, etc. The healthcare guidance system 1410 can adjust behavioral interventions based on input from the healthcare provider. For example, the healthcare provider can add health care support parameters, such as target goals for losing weight, reducing blood pressure, increasing exercise durations, etc., as well as constraints for optimization routines. The behavioral intervention programs can be modified by the user, healthcare provider, family member, authorized individual, etc.

The healthcare guidance system 1410 can forecast events, predict health states, and/or perform any of the techniques or methods disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; and PCT App. No. PCT/2021/028445. For example, the system 1400 can accurately determine the glucose concentration in the blood of an individual at a present time and/or in the future and can adaptively provide healthcare support to achieve health goals. The system 1400 can then develop personalized biomonitoring and/or provide personalized healthcare recommendations or information for the treatment of diabetes and other chronic conditions, exercise programs, or the like. Behavioral interventions programs can be used to further assist with user responsiveness.

In some embodiments, the system 1400 can provide each user 1402 with one or more self-care modes. The healthcare guidance system 1410 can collect health user data of the user 1402a and can identify a health metric based on the health data. The system 1410 then identifies a self-care mode from a plurality of self-care modes by, for example, analyzing a plurality of available self-care modes, each having one or more contributing factors, determining one or more predictions of the health care metric based on the self-care modes, identifying contribution factors of the self-care modes for the predictions, and selecting a self-care mode from the plurality of available self-care modes based on the contribution factors. The system 1400 can now put a notification for the user for viewing on a device of the user 1402a. The notification can include prompts, recommendations, self-care actions/programs, predictions, identification of contributing health factors, prompts, or other suitable data. Self-care modes can be recommended continuously or periodically at regular or irregular intervals or in response to an event (e.g., unwanted predicted event, user inputted event, etc.)

If the user 1402a is concerned about cardiovascular health, the system 1400 can determine a cardiovascular risk score and can recommend a self-care mode to reduce or minimize the cardiovascular risk score. The collected health data can include systolic blood pressure data, blood pressure variability, exercise data (e.g., exercise data collected via a wearable device, smart phone, user input, etc.), cholesterol levels, or combinations thereof. When a new user device is available, the system 1400 can pair with the newly available device and automatically collect new data that may provide indications suitable for determining the cardiovascular risk score. The system 1410 can receive and analyze the data to determine one or more predictions, such as predicting a minimization reduction of the cardiovascular score. The system 1410 can also identify contributing factors, including diet, exercise, weight of the user, or the like. In some embodiments, the system provides ranking of the contributing health factors to allow the user 1402a to prioritize recommendation adherence, as discussed in connection with FIG. 15.

The system 1400 concurrently provides other support to the user 1402b. If the user 1402b suffers from diabetes, the system 1400 can support a glucose management self-care mode. Blood glucose levels, food data, exercise data, sleep data, and other health data can be transmitted to the system 1410. The system 1410 can analyze the data to predict hypoglycemic events, hyperglycemic events, maximum/minimum blood glucose levels, blood glucose level ranges, or the like. The system 1410 can also identify contributing health factors for the blood glucose levels, including diet, weight, exercise, sleep, or the like. The system 1400 can generate recommendations and corresponding warnings, incentives, prompts, or other notifications. The system can also evaluate whether the user history indicates a higher likelihood of user compliance or suggestions, notifications, and other demonstrations to generate corresponding recommendations.

The system 1400 can monitor the contributing health factors over a period of time to adaptively update the self-care mode and recommendations. When the user 1402b completes an exercise routine, the system can notify the user of changes of contributing health factors. For example, if the user reaches a healthy weight, the system 1410 can notify the users that his/her weight is not a contributing factor for glucose management, thereby informing the user to focus on other contributing factors. When the user completes actions, the system 1400 can assign corresponding positive scores or weights to preceding actions. This allows behavioral intervention to be incorporated into the self-care mode and corresponding recommendations.

In another implementation, users 1402 may be recommended a weight management self-care mode. The system 1400 can collect weight data, body mass index, age, sex (e.g. male or female), or other health data. The guidance system 1410 can predict an increase of the user's weight based on contributing health factors. The system 1400 can recommend dietary changes, sleeping schedule, exercise schedule, and other contributing health factors. The system can also analyze other recommended self-care modes to provide consistent actions. For example, the system 1400 can prioritize predictions for a particular user. This allows contributing health factors to prioritize predictions to be recommended. For example, a user can be recommended both a weight management self-care mode and a glucose management self-care mode. Predictions for weight management self-care mode can be long term weight loss, whereas the predictions for the glucose management self-care can be short-term blood glucose levels. The healthcare guidance system 1410 can provide actions for the self-care mode to keep the user within acceptable blood glucose levels while also providing actions for the weight management self-care mode for weight loss.

The systems disclosed herein can use weighting algorithms, optimization functions, and other routines/functions for providing self-care modes for a group of users (e.g., family members), managing multiple conditions, or other like. In some embodiments, the systems can provide a self-care mode for multiple users. For example, related users may suffer from the same or similar conditions. The self-care mode can be designed to manage the condition(s), such as hypertension, obesity, etc. The allows users (e.g., family members, friends, partners, etc.) to implement a single self-care plan (including dietary program, dietary program, etc.) to improve overall health. The system can notify the users if a single self-care mode is no longer suitable to both users. The system can then send individualized self-care modes to each user.

A self-care mode can be designed for multiple conditions by, for example, using weighting, constrained optimization, averaging function, or the like. In non-dominant condition implementations, multiple conditions can be weighted, scored, and optimized to manage, for example, (1) diabetes and hypertension; (2) diabetes and cardiovascular disease; (3) diabetes and obesity; (4) diabetes, cardiovascular risk, and obesity; etc. A self-care mode for reducing cardiovascular risk can also manage hypertension and blood glucose levels. Predicted blood pressure and blood glucose levels can be weighted and prioritized based on, for example, prioritized user goals, healthcare provider input, healthcare impact score, etc. In some dominant condition implementations, one or more conditions can be identified for constraints used during optimization to reduce/increase one or more objectives as long as another health objective for the dominant condition is above or below a certain threshold. This allows important health conditions to be prioritized.

FIG. 15 is a schematic diagram illustrating an embodiment of the system for providing adaptive healthcare support for a user 1402, in accordance with an embodiment of the present technology. The description of the system 1400 of FIG. 14 applies equally to the system 1500 unless indicated otherwise, and system 1400 can perform the methods disclosed herein.

The system 1500 can collect user data, user input, auxiliary data, etc. The user data can be collected by sensors (e.g., glucose sensors, wearable sensors, etc.), received from a remote computing device (e.g., a cloud platform storing user history data, real-time data, etc.), or other data. The user input can be health data (e.g., weight, BMI, etc.), dietary data, exercise or motion data (e.g., distance walked, distance run, etc.), goals, achievements, ratings/rankings (e.g., ranked goals, rated activities, etc.), or other data inputted by the user using one or more computing devices, such as a mobile phone, computer, etc. This allows a user to input data that is not automatically collected. The auxiliary data 1516 can be selected by the system 1410 to modify the adaptive support machine-learning model based on received indication of the response. The auxiliary data 1416 can include predictions (e.g., short-term predictions, long-term predictions, forecasted events, etc.), environment data (e.g., weather data, temperature data, etc.), or the like. The auxiliary data 1516 can be inputted to models to generate output data based on non-user specific parameters.

The system 1410 can request auxiliary data or communicate with device(s) to receive data indicative of a past user state, a past action presented to the user, a past user behavior, health status, or combinations thereof. In some embodiments, the system 1410 can establish communication with connected device (e.g., vehicle) associated with the user, IoT hubs (e.g., IoT devices with Google Assistance, Siri, Alexa, etc.), IoT devices (e.g., motion sensors, cameras, etc.), surveillance systems, etc. For example, when a user arrives home after work, the user may not be receptive to certain prompts for a period of time. The system 1410 can receive auxiliary data (e.g., a garage door opening, surveillance system turned OFF, etc.) indicating when the user returned home. The system 1410 can determine a program or a set of delivery details for adjusting a content and/or a delivery timing for recommended actions based on the user's arrival time. The system 1410 can adaptively request and receive data from different sources to adaptively train the models and engines disclosed herein. The system 1410 can manage identification and authentication for integration with auxiliary platforms, devices, and systems. In some applications, the system 1410 can incorporate weather data to maximize behavior intervention by, for example, providing prompts (e.g., prompts to exercise outside, walk, etc.) suitable for the weather conditions. Health predictions can be considered to develop behavioral interventions designed to increase health scores for the user, enhance goal setting, and accurately identify self-care modes or user states.

The user input 1514 can include one or more new goals, such as maintaining glucose levels, losing weight within a set period or time, answers to questions, risk scores, etc. The guidance system 1410 can select databases (e.g., pooled user data) and models for recommending user device(s) for collecting target data, analyzing the one or more new goals, recommending user device(s) for reinforcements, etc. The guidance system 1410 can send the information (e.g., future health metrics, self-care modes, alternative self-care modes, etc.) to user device 1532 for viewing by a healthcare provider or third-party device 1538, or third-party device 1420 as discussed in connection with FIG. 14.

The system 1410 can receive one or more user history items associated with the user 1402. The user history items can define a past user state, a past action presented to the user, a past user behavior, or combinations thereof. The system 1410 can select an adaptive healthcare support engine 1522 trained to estimate user information, such a current state or predicted state of the user, based on the one or more user history items. The system 1320 can utilize the adaptive healthcare support engine 1522 or another engine 1524 to identify one or more actions for the user based on the user information. The user device(s) 1518, 1532 can execute the one or more identified actions for the user and can receive an indication of a behavior of the user performed in response to the action. The system 1410 can update one or more of the adaptive support models (e.g., models 1522, 1524, 1526, etc.) based on the received indication of the behavior detected by the user devices 1518 or 1432, or indicated by the user.

In some embodiments, the system 1410 can receive new data from the user 1302. The new data can represent health sensor data, a biometric condition, user input data, a user motion, a user location, or a combination thereof. The health sensor data from a user device 1518 can include glucose levels, blood pressure, heart rate, analyte levels, or other detectable indicators of the state of the user. The system 1410 can access one or more user history items (e.g., items stored in database 1426) defining at least one of a past user state, a past action presented to the user, and a past user behavior. The past user state can represent a physiological or a health condition of the user occurring or processed at a past time. The past action can represent a previously identified action taken by the user. The past user behavior can represent a repeated action occurring with a temporal pattern. The actions can be detected or identified by user device(s) 1518, 1532, or other suitable means, such as biomonitoring devices or via user input 1514.

The system 1410 can estimate a recent state of the user based on the new data and the one or more user history items. The recent state represents a current or a recent health condition of the user (e.g., most recent health condition, health condition within a predetermined period of time, etc.). The health condition can be, for example, hypoglycemic state, hyperglycemic state, high blood pressure, etc. The system 1410 can determine a likely outcome (e.g., increase/decrease in glucose levels, blood pressure, etc.) based on the recent state for represent a thresholding health condition of the user likely to occur at a future time. The system 1410 can then identify one or more actions for the user based on the recent state using one or more adaptive support machine-learning models. The actions can be sent to the user devices 1532 for user notification to affect a targeted user action before the future time to prevent or adjust the likely outcome. In some embodiments, the identified actions are selected based on whether the user devices 1532 is capable of identifying the action. For example, if the user has wearable exercise monitor, the identified actions can include exercises detectable by the wearable exercise monitor. In some embodiments, the user can be prompted to input whether the action has been completed. The system 1410 can also provide goal(s) 1534, output data 1536, or other information disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; and PCT App. No. PCT/2021/028445, which are herein incorporated by reference in their entireties. For example, the goal(s) can include food-related health goals, includes diabetes management, weight management, increased fertility, reduction of alcohol consumption, or other user inputted goals. The output data 1536 can include a personalized health report that includes one or more food diaries, user-specific health predictions, user-specific health recommendations, or user-specific biometric summaries (e.g., glucose levels, analyte levels, predictions, etc.), or other information disclosed herein.

The system 1410 can also determine a set of delivery details for adjusting a content and/or a delivery timing for the recommended action. The user device(s) 1532 can execute the identified action according to the set of delivery details. The system 1500 can receive or identify and indication of a response of the user performed in response to the action. When the response corresponds to the past user behavior, the system 1410 can update associated adaptive support machine-learning models based on the received indication of the response. The system 1410 can add engines and models based on newly available data, new users, or the like to provide adaptability.

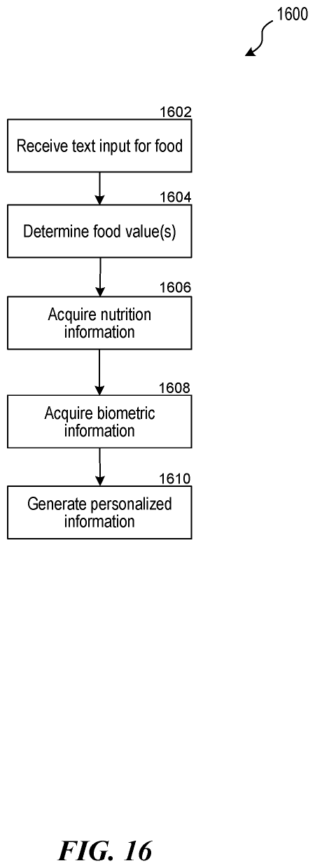

FIG. 16 is a flow diagram illustrating a process 1600 for providing health information, in accordance with embodiments of the present technology. In some implementations, process 1600 is triggered by a user activating a subscription for a food logging service, inputting food information into an application, or the user downloading an application on a device for food logging. In various implementations, process 1600 is performed locally on the user device or performed by cloud-based device(s) that can support food logging.

At step 1602, process 1600 receives user textual input (e.g., user input 1514 of FIG. 15) for food (or beverage) from a user device (e.g., user device(s) 1518 of FIG. 15). For example, a user can enter (e.g., by typing the food information into the food logger or by using a speech-to-text application) the information of consumed food (e.g., food/beverages the user has consumed or may consume).

At step 1604, process 1600 determines quantified food value(s) for the consumed food based on the user textual input (Process 200 of FIG. 2). The quantified food value(s) can include a serving, a portion, or an amount of food. For example, the quantified values can include measurements such as a teaspoon, a cup, a quart, etc. At step 1606, process 1600 acquires nutrition information for the quantified food value(s), as described in FIGS. 4-10.

At step 1608, process 1600 acquires biometric information (e.g., user data 1512 of FIG. 15) of the user associated with the food. For example, process 1600 retrieves biometric information such as blood pressure, heart rate, BMI, body temperature, etc. of the user. Process 1600 can acquire the biometric data from a user device, such as user device(s) 104 (biosensors 104a, mobile device(s) 104b, and/or wearable device(s) 104c) of FIG. 1.

At step 1610, process 1600 generates personalized health information for the user based on the acquired nutrition information and the biometric data. The personalized health information can relate the food to the health of the user. For example, the personalized health information includes a food diary, health information of the user, user specific health prediction, user-specific health recommendation, user-specific biometric summary, and/or self-care information for the user. The user can access the personalized information through the food logger application on a device (e.g., user device(s) 1532 of FIG. 15). The food diary can include food information, such as the type, amount, ingredients, nutrition, and/or the time the food was consumed.

Process 1600 can determine a relationship of the food to the health of the user based on a predicted health event and/or real-time health state of the user. For example, if the user is diabetic, process 1600 can predict that the ingredients in the food can cause the user's blood sugar levels to approach levels that can cause negative health effects. In some implementations, process 1600 uses a biometric trained machine-learning engine to determine the relationship between biometric data of the user and the consumed food.

Process 1600 can analyze the personalized health information to generate an output (e.g., output 1536 of FIG. 15) of a user-specific health prediction, a user-specific health recommendation, and/or a user-specific biometric summary for viewing by the user to manage health and/or self-care, such as goals 1534 of FIG. 15. Process 1600 can determine whether an adverse health criterion (e.g., blood sugar outside of a range, heart rate above or below a range, etc.) for the user is met based on the personalized health information. When the adverse health criterion is met, process 1600 can perform a corrective health action for the user. The corrective health action can include sending a corrective action notification (e.g., text, email, alert, health prediction, or sequence of steps for the corrective action) to the user or generating a personalized health report for the user indicating one or more outcomes associated with the logging of consumed food. The corrective health action can include sending commands to a wearable device (e.g., insulin pump), an implant (e.g., pacemaker), or the like. For example, process 1600 can generate and send the user the health report to notify the user of health risks associated with consuming the food. In some implementations, process 1600 can send the biometric data or a portion of the biometric data to a healthcare device of the user. In some cases, the healthcare device automatically provides care to the user.

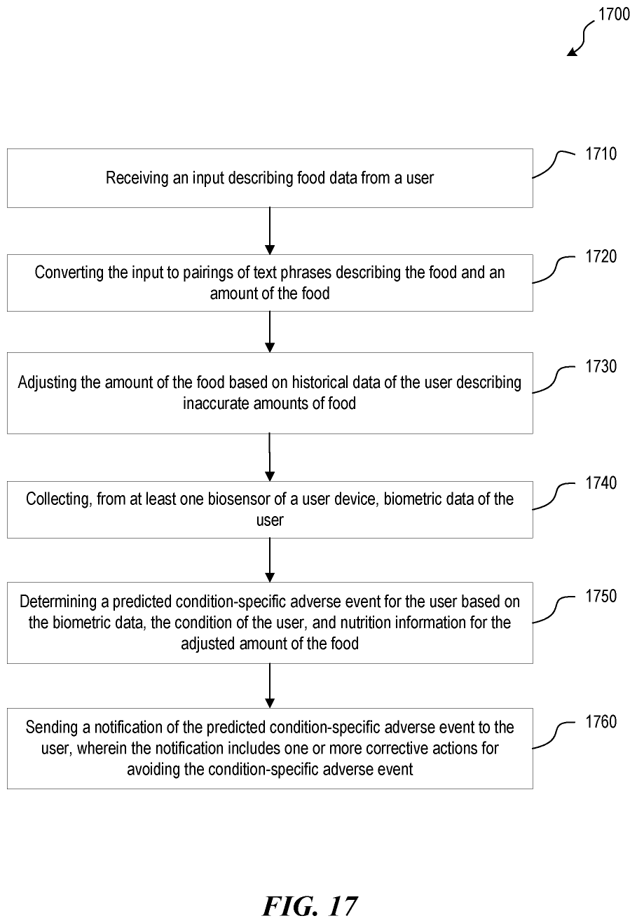

FIG. 17 is a flowchart illustrating a process 1700 for identifying events (e.g., condition-specific adverse events) for dietary health, in accordance with embodiments of the present technology. In some implementations, a machine-learning platform performs process 1700 and can use condition-specific (e.g., Type 1 or Type 2 diabetes, blood disorder condition, blood-pressure related condition, etc.) machine learning modules to be applied to biometric data of the user and food data to predict adverse events of the user post-consumption of food based on a condition (e.g., diabetic state, a hypoglycemic state, a hyperglycemic state, a high blood pressure state, a low blood pressure state, etc.) of the user. The machine-learning condition-specific modules can be trained using user data of users with those specific conditions. For example, hypoglycemic/hypoglycemic machine-learning modules can be trained using user data from users prone to experiencing hypoglycemic/hypoglycemic events. In some embodiments, different hypoglycemic machine-learning modules and hypoglycemic machine-learning modules can be used. The machine-learning modules can be applied to data (e.g., biometric data of the user, food data, etc.) to predict adverse events, such as post-consumption events.

At step 1710, process 1700 receives an input (e.g., textual input, audio input, etc.) describing food data that a user has consumed or is about to consume. At step 1720, process 1700 converts the input to pairings of text phrases describing the food and an amount of the food.

At step 1730, process 1700 adjusts/scales the amount of the food based on historical data of the user describing inaccurate amounts of food. The machine learning platform can be trained to identify that the user underestimates or overestimates amounts of food in their meal. For example, if the user's blood sugar values are impacted to a greater degree than the amount of food that the user describes, process 1700 can determine the user underestimates the amount of food.

Process 1700 provides portion training protocols for user education to the user. For example, if the user's descriptions of their food are inaccurate for a time threshold, process 1700 can alert the user of the inaccuracies of their food descriptions. Portion estimation feedback can be provided to the user to train the user's portion estimation skills. Those skills can be scored over time to provide accuracy feedback.

At step 1740, process 1700 collects (e.g., from at least one biosensor of a user device), biometric data of the user. Process 1700 can analyze the nutrition information of the food to generate food values. Process 1700 can generate a personalized health report based on a relationship between the collected biometric data and nutrition information. The personalized health report can include a food diary, a user-specific health prediction, a user-specific health recommendation, or a user-specific biometric summary. The user device can display the health report to the user.

At step 1750, process 1700 determines a predicted condition-specific adverse event (e.g., hypoglycemic event, a hyperglycemic event, a ketosis event, a cardiovascular event, etc.) for the user based on the biometric data, the condition of the user, and/or nutrition information for the adjusted amount of the food.

At step 1760, process 1700 sends sending a notification of the predicted condition-specific adverse event to the user. The notification can include one or more corrective actions for the user to avoid the condition-specific adverse event. The corrective action can include, for example, administration of medication, such as insulin for managing diabetes, noninsulin medication for managing Type 1 and/or Type 2 diabetes, weight loss drugs, etc. The correction active may include a medical session, such as blood letting for hemochromatosis, hemodialysis for kidney failure, etc. The corrective action may also include dietary recommendations for mitigating the condition, such as reducing red meat or iron consumption for hemochromatosis). Multiple corrective actions can be ranked and include projected outcomes.

Process 1700 can send notifications to family members or shared users (e.g., parents, relatives, etc.) regarding predicted condition-specific adverse events of the user. In some implementations, process 1700 synchronizes shared tracking accounts to assist the use in reaching health or fitness goals. For example, process 1700 can provide dietary suggestions to the user to assist the user in maximizing performance in sporting events, workouts, weight loss or weight gain.

FIG. 18 illustrates an example of a user interface 1800 displaying health information 1802 (e.g., blood pressure metrics, heartrate metrics, body temperature metrics, blood sugar alerts, etc.). The user interface 1800 can display a progress indicator 1804 which illustrates the progress of a user in reaching a goal. User interface 1800 can display the notifications of the predicted condition-specific adverse events as described in FIG. 17 and described herein. The user interface 1800 can also display information discussed in connection with FIG. 8. For example, meal information can be correlated to the health data 1802. Other information disclosed herein can be incorporated into displayed information of the user interface 1800.

### CONCLUSION

The embodiments set forth in the foregoing description do not represent all embodiments consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the embodiments described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other embodiments can be within the scope of the following claims.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

As used herein, the phrase “and/or” as in “A and/or B” refers to A alone, B alone, and A and B.

As used herein, the term “user” can refer to any entity including a person or a computer.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

Furthermore, the skilled artisan will recognize the interchangeability of various features from different embodiments disclosed herein and disclosed in U.S. Pat. No. 63/034,331; U.S. Pat. Nos. 9,008,745; 9,182,368; 10,173,042; U.S. application Ser. No. 15/601,204 (US Pub. No. 2017/0251958); U.S. application Ser. No. 15/876,678 (U.S. Pub. No. 2018/0140235); U.S. application Ser. No. 14/812,288 (US Pub. No. 2016/0029931); U.S. application Ser. No. 14/812,288 (US Pub. No. 2016/0029966); US Pub. No. 2017/0128009; U.S. App. No. 62/855,194; U.S. App. No. 62/854,088; U.S. App. No. 62/970,282; U.S. App. No. 63/188,641; PCT App. No. PCT/US19/49270 (WO2020/051101), U.S. application Ser. No. 17/236,753; PCT App. No. PCT/2021/028445; and U.S. application Ser. No. 17/338,586, which are all incorporated by reference in their entireties. For example, methods of detection, sensors, detection elements, biosensors, user devices, etc. can be incorporated into or used with the technology disclosed herein. All of patent and applications referenced are incorporated herein by reference in their entireties. Similarly, the various features and acts discussed above, as well as other known equivalents for each such feature or act, can be mixed and matched by one of ordinary skill in this art to perform methods in accordance with principles described herein. All of the above cited applications and patents are herein incorporated by reference in their entireties.

From the foregoing, it will be appreciated that specific embodiments of the invention have been described herein for purposes of illustration, but that various modifications may be made without deviating from the scope of the invention. Accordingly, the invention is not limited except as by the appended claims.

1. A computer-implemented method for using biosensor data to identify condition-specific adverse events for dietary health, the method comprising:
receiving, at a machine-learning platform, an input describing food data from a user,
wherein the machine-learning platform includes a plurality of condition-specific machine learning modules to be applied to biometric data of the user and food data to predict adverse events of the user post-consumption of food based on a condition of the user;
converting the input to pairings of text phrases describing the food and an amount of the food;
adjusting, by the machine-learning platform, the amount of the food based on historical data of the user describing inaccurate amounts of food;
collecting, from at least one biosensor of a user device, biometric data of the user;
determining a predicted condition-specific adverse event for the user based on the biometric data, the condition of the user, and nutrition information for the adjusted amount of the food; and
sending a notification of the predicted condition-specific adverse event to the user, wherein the notification includes one or more corrective actions for avoiding the condition-specific adverse event.

2. The computer-implemented method of claim 1, further comprising:
analyzing the nutrition information to generate one or more food values;
identifying the biometric data of the user associated with consuming the food;
determining at least one relationship between the identified biometric data and the adjusted amount of the food;
generating a personalized health report based on the at least one relationship, wherein the personalized health report includes at least one of:
a food diary,
a user-specific health prediction,
a user-specific health recommendation, or
a user-specific biometric summary; and
providing the personalized health report for viewing by the user for managing health and/or self-care.

3. The computer-implemented method of claim 1, further comprising:
receiving one or more user health goals;
using the machine-learning platform to determine at least one relationship between the biometric data of the user and the adjusted amount of the food; and
selecting personalized information to be included in a personalized health report based on the one or more user health goals, wherein the personalized health report includes the selected personalized information.

4. The computer-implemented method of claim 1, further comprising:
determining whether the predicted condition-specific adverse event meets or exceeds at least one adverse health criterion for the user; and
in response to determining that the at least one adverse health criterion for the user is met, performing one or more of:
sending the notification to the user, wherein the notification includes at least one of an alert, a health prediction, or sequence of steps for the one or more corrective health actions;
sending at least a portion of the biometric data to a healthcare device of the user, wherein the healthcare device is configured to automatically provide care to the user, wherein the healthcare device includes a wearable device or an implant device; and
generating a personalized health report for the user indicating one or more outcomes associated with logging of consumed food.

5. The computer-implemented method of claim 1, further comprising:
identifying a location of the user;
performing one or more pre-meal routines to provide dietary suggestions, track dietary effects to the user, and provide a progress of user goals; and
sending one or more notifications to the user to assist with ordering food at the location.

6. The computer-implemented method of claim 1, further comprising:
acquiring nutrition information for the food based on the text phrases; and
scaling the acquired nutrition information according to a syntax of the input and at least one quantifier from the input of the user.

7. The computer-implemented method of claim 1,
wherein the condition includes at least one of a diabetic state, a hypoglycemic state, a hyperglycemic state, a high blood pressure state, or a low blood pressure state, and
wherein the condition-specific adverse event includes at least one of a hypoglycemic event, a hyperglycemic event, a ketosis event, or a cardiovascular event.

8. A system comprising:
one or more processors; and
one or more memories storing instructions that, when executed by the one or more processors, cause the system to perform a process for identifying condition-specific adverse events for dietary health, the process comprising:
receiving, at a machine-learning platform, an input describing food data from a user,
wherein the machine-learning platform includes a plurality of condition-specific machine learning modules to be applied to biometric data of the user and food data to predict adverse events of the user post-consumption of food based on a condition of the user;
converting the input to pairings of text phrases describing the food and an amount of the food;
adjusting, by the machine-learning platform, the amount of the food based on historical data of the user describing inaccurate amounts of food;
collecting, from at least one biosensor of a user device, biometric data of the user;
determining a predicted condition-specific adverse event for the user based on the biometric data, the condition of the user, and nutrition information for the adjusted amount of the food; and
sending a notification of the predicted condition-specific adverse event to the user, wherein the notification includes one or more corrective actions for avoiding the condition-specific adverse event.

9. The system according to claim 8, wherein the process further comprises:
analyzing the nutrition information to generate one or more food values;
identifying the biometric data of the user associated with consuming the food;
determining at least one relationship between the identified biometric data and the adjusted amount of the food;
generating a personalized health report based on the at least one relationship, wherein the personalized health report includes at least one of:
a food diary,
a user-specific health prediction,
a user-specific health recommendation, or
a user-specific biometric summary; and
providing the personalized health report for viewing by the user for managing health and/or self-care.

10. The system according to claim 8, wherein the process further comprises:
receiving one or more user health goals;
using the machine-learning platform to determine at least one relationship between the biometric data of the user and the adjusted amount of the food; and
selecting personalized information to be included in a personalized health report based on the one or more user health goals, wherein the personalized health report includes the selected personalized information.

11. The system according to claim 8, wherein the process further comprises:
determining whether the predicted condition-specific adverse event meets or exceeds at least one adverse health criterion for the user; and
in response to determining that the at least one adverse health criterion for the user is met, performing one or more of:
sending the notification to the user, wherein the notification includes at least one of an alert, a health prediction, or sequence of steps for the one or more corrective health actions;
sending at least a portion of the biometric data to a healthcare device of the user, wherein the healthcare device is configured to automatically provide care to the user, wherein the healthcare device includes a wearable device or an implant device; and
generating a personalized health report for the user indicating one or more outcomes associated with logging of consumed food.

12. The system according to claim 8, wherein the process further comprises:
identifying a location of the user;
performing one or more pre-meal routines to provide dietary suggestions, track dietary effects to the user, and provide a progress of user goals; and
sending one or more notifications to the user to assist with ordering food at the location.

13. The system according to claim 8, wherein the process further comprises:
acquiring nutrition information for the food based on the text phrases; and
scaling the acquired nutrition information according to a syntax of the input and at least one quantifier from the input of the user.

14. The system according to claim 8,
wherein the condition includes at least one of a diabetic state, a hypoglycemic state, a hyperglycemic state, a high blood pressure state, or a low blood pressure state, and
wherein the condition-specific adverse event includes at least one of a hypoglycemic event, a hyperglycemic event, a ketosis event, or a cardiovascular event.

15. A non-transitory computer-readable medium storing instructions that, when executed by a computing system, cause the computing system to perform operations for identifying condition-specific adverse events for dietary health, the operations comprising:
receiving, at a machine-learning platform, an input describing food data from a user,
wherein the machine-learning platform includes a plurality of condition-specific machine learning modules to be applied to biometric data of the user and food data to predict adverse events of the user post-consumption of food based on a condition of the user;
converting the input to pairings of text phrases describing the food and an amount of the food;
adjusting, by the machine-learning platform, the amount of the food based on historical data of the user describing inaccurate amounts of food;
collecting, from at least one biosensor of a user device, biometric data of the user;
determining a predicted condition-specific adverse event for the user based on the biometric data, the condition of the user, and nutrition information for the adjusted amount of the food; and
sending a notification of the predicted condition-specific adverse event to the user, wherein the notification includes one or more corrective actions for avoiding the condition-specific adverse event.

16. The non-transitory computer-readable medium of claim 15, wherein the operations further comprise:
analyzing the nutrition information to generate one or more food values;
identifying the biometric data of the user associated with consuming the food;
determining at least one relationship between the identified biometric data and the adjusted amount of the food;
generating a personalized health report based on the at least one relationship, wherein the personalized health report includes at least one of:
a food diary,
a user-specific health prediction,
a user-specific health recommendation, or
a user-specific biometric summary; and
providing the personalized health report for viewing by the user for managing health and/or self-care.

17. The non-transitory computer-readable medium of claim 15, wherein the operations further comprise:
receiving one or more user health goals;
using the machine-learning platform to determine at least one relationship between the biometric data of the user and the adjusted amount of the food; and
selecting personalized information to be included in a personalized health report based on the one or more user health goals, wherein the personalized health report includes the selected personalized information.

18. The non-transitory computer-readable medium of claim 15, wherein the operations further comprise:
determining whether the predicted condition-specific adverse event meets or exceeds at least one adverse health criterion for the user; and
in response to determining that the at least one adverse health criterion for the user is met, performing one or more of:
sending the notification to the user, wherein the notification includes at least one of an alert, a health prediction, or sequence of steps for the one or more corrective health actions;
sending at least a portion of the biometric data to a healthcare device of the user, wherein the healthcare device is configured to automatically provide care to the user, wherein the healthcare device includes a wearable device or an implant device; and
generating a personalized health report for the user indicating one or more outcomes associated with logging of consumed food.

19. The non-transitory computer-readable medium of claim 15, wherein the operations further comprise:
identifying a location of the user;
performing one or more pre-meal routines to provide dietary suggestions, track dietary effects to the user, and provide a progress of user goals; and
sending one or more notifications to the user to assist with ordering food at the location.

20. The non-transitory computer-readable medium of claim 15,
wherein the condition includes at least one of a diabetic state, a hypoglycemic state, a hyperglycemic state, a high blood pressure state, or a low blood pressure state, and
wherein the condition-specific adverse event includes at least one of a hypoglycemic event, a hyperglycemic event, a ketosis event, or a cardiovascular event.

21.-64. (canceled)
